(19) United States
(12) Patent Application Publication
(10) Pub. No.: US 2026/0193305 A1
(43) Pub. Date: Jul. 9, 2026

(54) COMPOSITIONS COMPRISING DIMERIZED CXCL 10-DERIVED PEPTIDES AND METHODS OF USE THEREOF

(71) Applicant: University of Virginia Patent Foundation, Charlottesville, VA (US)

(72) Inventors: Molly A. HUGHES, Charlottesville, VA (US); Matthew CRAWFORD, Charlottesville, VA (US)

(21) Appl. No.: 19/370,846

(22) Filed: Oct. 28, 2025

Related U.S. Application Data
(60) Provisional application No. 63/713,000, filed on Oct. 28, 2024.

(51) Int. Cl. C07K 14/52; A61L 26/00

(52) U.S. Cl. CPC C07K 14/521; A61L 26/0066; A61L 2300/406

(57) ABSTRACT

Provided are peptide dimers, including dimers from modified peptides, that have antibacterial activity, including against multidrug-resistant bacteria. In some embodiments, the peptides that form the dimers include the amino acid sequence RTVRCTCI or amino acid substitution variants thereof that dimerize via cysteine-based disulfide bonds. The peptide dimers can be polymer-functionalized, encapsulated in a particle, embedded in and/or on a solid support, impregnated in a dressing, and/or is formulated for use in a nebulizer, for topical administration, and/or for systemic administration. Also provided are medical devices having an antibacterial agent that includes one or more of the presently disclosed peptide dimers embedded in and/or associated with a support layer, and methods for inhibiting growth of and/or killing bacteria, for treating or preventing community and/or nosocomial infections, for treating bacterial infections present in wounds, for treating pulmonary infections, for treating or preventing systemic bacterial infections, and for combination therapies with conventional antibiotics.

### CROSS-REFERENCE TO RELATED APPLICATION[S]

This application claims priority to, and the benefit of, U.S. Provisional Patent Application Ser. No. 63/713,000, entitled “COMPOSITIONS COMPRISING DIMERIZED CXCL10-DERIVED PEPTIDES AND METHODS OF USE THEREOF” and filed on Oct. 28, 2024, the entire contents of which are incorporated herein by reference as if set forth in its entirety.

### STATEMENT REGARDING FEDERALLY SPONSORED RESEARCH OR DEVELOPMENT

This invention was made with government support under Grant No. AI150941 awarded by National Institutes of Health. The Government has certain rights in the invention.

### SEQUENCE LISTING

The instant application contains a Sequence Listing which has been submitted electronically in .XML format and is hereby incorporated by reference in its entirety. Said .XML copy, created on Mar. 23, 2026, is named “222117_1680_sequence_listing.xml” and is 426,265 bytes in size.

### TECHNICAL FIELD

The presently disclosed subject matter relates to compositions and methods useful for treating and preventing bacterial infections. In particular, the presently disclosed subject matter relates to compositions comprising dimerized peptides with enhanced bactericidal activity that can be administered to subject to treat and/or prevent bacterial infections.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to peptide dimers. In some embodiments, the peptide dimers comprise, consist essentially of, or consist of a first peptide and a second peptide, wherein
(i) the first and second peptides both comprise, consist essentially of, or consist of an amino acid sequence selected from the group consisting of RTVRCTCI (SEQ ID NO: 1), RFVRCTCI (SEQ ID NO: 14), RWVRCTCI (SEQ ID NO: 18), RYVRCTCI (SEQ ID NO: 19), RTVRCRCI (SEQ ID NO: 26), RTVRCMCI (SEQ ID NO: 37), RYVRCRCI (SEQ ID NO: 59), RRVRCRCI (SEQ ID NO: 85), RWVRCWCI (SEQ ID NO: 108), and modified peptides thereof, wherein the modified peptides comprise one or more amino acid substitutions with the proviso that the first and second peptides both have at least one and optionally two or more cysteines; and
(ii) optionally wherein one or more and optionally all of the amino acids of the first peptide, the second peptide, or both, are D-amino acids.

In some embodiments, the peptide dimer has bactericidal and/or bacteriostatic activity against a bacterium selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to Salmonella enterica serovars such as but not limited to Salmonella enterica serovar Typhi and Shigella flexneri; and biothreat agents such as but not limited to Bacillus anthracis in both vegetative and spore forms. In some embodiments, the bacterium is an MDR strain of Enterococcus faecium, Staphylococcus aureus, Klebsiella pneumoniae, Acinetobacter baumannii, Pseudomonas aeruginosa, Salmonella enterica, optionally Salmonella enterica serovar Typhi, or Shigella flexneri. In some embodiments, the first and/or the second peptide comprises, consists essentially of, or consists of the amino acid sequence RTVRCTCI (SEQ ID NO: 1), RTVRCRCI (SEQ ID NO: 26), RTVRCMCI (SEQ ID NO: 37), or RYVRCRCI (SEQ ID NO: 59), or a modified amino acid sequence thereof, wherein the modified amino acid sequence comprises one or more amino acid substitutions, with the proviso that the first and second peptides both have at least one and optionally two or more cysteines; and optionally wherein one or more and further optionally all of the amino acids of the first peptide, the second peptide, or both, are D-amino acids. In some embodiments, the first peptide, the second peptide, or both is polymer-functionalized, encapsulated in a particle, embedded in and/or on a solid support, optionally wherein the peptide dimer is formulated for release from the solid support, impregnated in a dressing, optionally wherein the peptide dimer is formulated for release from the dressing, and/or is formulated for use in a nebulizer, for topical administration, and/or for systemic administration. In some embodiments, the first peptide and/or the second peptide is a modified peptide that comprises a modification at the N-terminus, the C-terminus, both the N-terminus and the C-terminus, and/or at an internal amino acid position including but not limited to amino acid position 2, optionally wherein one or more of the modifications are selected from the group consisting of an addition or an aminohexanoic acid (AHX), an azido alanine (Ala(N₃)), a propargylglycine (PRA), a photoaffinity label, optionally a benzoylbenzoic acid (4-BBA), an azido phenylalanine (Phe(4-N₃)), a diazirine-containing amino acid, optionally leucine, methionine, lysine, proline, or phenylalanine; and/or is a replacement of a phenylalanine with an azido phenylalanine (Phe(4-N₃)). In some embodiments, the first and second peptides are present in an anti-parallel orientation.

The presently disclosed subject matter also relates in some embodiments to pharmaceutical compositions comprising, consisting essentially of, or consisting of one or more peptide dimers as disclosed herein and a pharmaceutically acceptable carrier, diluent, or excipient. In some embodiments, the pharmaceutical composition is pharmaceutically acceptable for use in a human.

The presently disclosed subject matter also relates in some embodiments to medical devices comprising a support layer with an antibacterial agent embedded therein or associated therewith. In some embodiments, the antibacterial agent comprises one or more peptide dimers as disclosed herein. In some embodiments, the medical device is a wound dressing. In some embodiments, one or more of the peptide dimers are encapsulated in a particle that is embedded in or associated with the support layer.

The presently disclosed subject matter also relates in some embodiments to methods for inhibiting the growth of and/or killing bacteria. In some embodiments, the methods comprise, consist essentially of, or consist of contacting the bacteria with an effective amount of an antibacterial agent comprising, consisting essentially of, or consisting of at least one peptide dimer as disclosed herein. In some embodiments, the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to a Salmonella enterica serovars such as but not limited to Salmonella enterica serovar Typhi and Shigella flexneri; and biothreat agents such as but not limited to Bacillus anthracis in both vegetative and spore forms.

The presently disclosed subject matter also relates in some embodiments to methods for treating and/or preventing community and/or nosocomial infections in subjects. In some embodiments, the methods comprise, consist essentially of, or consist of administering to a subject a composition comprising at least one peptide dimer as disclosed herein. In embodiments, an effective can be in the low microgram per milliliter range (e.g., 4-64 ug/mL). In embodiments, an effective about (for example, for In vivo animal models or other in vivo administration), an effective amount can be in the low milligram per kilogram range. For example, in certain embodiments, an effective amount can be about 5 mg/kg, about 10 mg/kg, or about 2.5 mg/kg administered topically or by systemic injection (intraperitoneal) once or twice a day.

The presently disclosed subject matter also relates in some embodiments to methods for treating bacterial infections present in wounds. In some embodiments, the methods comprise, consist essentially of, or consist of contacting the wound with an effective amount of a composition comprising one or more peptide dimers as disclosed herein.

The presently disclosed subject matter also relates in some embodiments to methods for treating pulmonary infections in subjects. In some embodiments, the methods comprise, consist essentially of, or consist of administering to a subject in need thereof an effective amount of a composition comprising one or more peptide dimers as disclosed herein. In some embodiments, the composition is administered to the subject intranasally, by inhalation, optionally wherein the one or more peptides in the composition is/are aerosolized, or a combination thereof. Administration of embodiments of peptides described herein may also be via intraperitoneal delivery or other systemic delivery (e.g., intravenous, intramuscular, or subcutaneous delivery).

The presently disclosed subject matter also relates in some embodiments to methods for treating or preventing systemic bacterial infections in subjects. In some embodiments, the methods comprising administering to a subject in need thereof an effective amount of a composition comprising one or more peptide dimers of the presently disclosed subject matter. In some embodiments, the presently disclosed methods further comprise administering to a subject a conventional antibiotic.

The presently disclosed subject matter also relates in some embodiments to methods for inhibiting the growth of biofilms. In some embodiments, the methods comprise, consist essentially of, or consist of contacting a biofilm with an effective amount of an antibacterial agent comprising, consisting essentially of, or consisting of at least one peptide dimer of the presently disclosed subject matter.

The presently disclosed subject matter also relates in some embodiments to uses of the peptide dimers of the presently disclosed subject matter for preventing and/or treating bacterial infections.

The presently disclosed subject matter also relates in some embodiments to compositions comprising, consisting essentially of, or consisting of at least one peptide dimer of the presently disclosed subject matter.

Accordingly, in some embodiments the presently disclosed subject matter relate to uses of the dimerized peptides, conjugates thereof, and/or any combination thereof, for preventing or treating bacterial infections.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Detailed Description and Figures.

### BRIEF DESCRIPTION OF THE FIGURES

FIG. 1 shows mass spectrometry traces of D8, which was oxidized and then treated+/−Tris(2-carboxyethyl)phosphine (TCEP).

FIG. 2 depicts HPLC traces of unguided oxidation showing the separation of 2 distinct D8 dimers by HPLC.

FIG. 3 shows mass spectrometry traces confirming the successful synthesis of anti-parallel and parallel dimeric forms of D8.

FIG. 4 shows the minimum inhibitory concentrations (MICs) of the two dimeric forms of peptide D8 that were determined against Klebsiella pneumoniae strain ATCC 43816.

FIG. 5 is a graph showing antimicrobial activity of the anti-parallel dimer (P201) against K. pneumoniae strain ATCC 43816.

FIG. 6 is a graph showing the effects of different culture media on the bactericidal efficacy of the anti-parallel dimer (P201).

FIG. 7 shows photographs of the determination of MICs against Acinetobacter baumannii strain 0088. Anti-parallel D8 is shown in the top panel whereas parallel D8 is shown in the bottom panel.

FIG. 8 shows photographs of the determination of MICs against Acinetobacter baumannii Strain 0282. Anti-parallel D8 is shown in the top panel whereas parallel D8 is shown in the bottom panel.

FIG. 9 is a table depicting MICs of anti-parallel D8 (P201) and parallel D8 (P261) against various wild type (WT) and antibiotic-resistant bacteria.

FIG. 10 depicts results of a human red blood cell hemolysis assay. To assess potential hemolytic activity, the anti-parallel dimer (P201) and the parallel dimer (P261) were tested in a human red blood cell (RBC) hemolysis assay—a standard in vitro method that measures peptide-induced RBC rupture and hemoglobin release by spectrophotometry.

FIG. 11 is a graph of human cell cytotoxicity of the anti-parallel dimer (P201) and the parallel dimer (P261) that were tested using the lung epithelial cell line A549 and a dye-based in vitro cytotoxicity assay where fluorescent signal is proportional to cell death FIG. 12 shows representative phase-contrast images of anti-parallel dimer (P201) and/or parallel dimer (P261) against B. anthracis Sterne strain spores that were treated with 50 μM of individual peptides.

FIG. 13 is a graph of colony counts from the experiment of FIG. 12 above.

FIG. 14 is a photograph of representative samples of activities of D8 dimers with bioorthogonal “click-chemistry” labels.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. General Considerations

Chemokines are chemotactic cytokines that are important regulators of leukocyte-mediated inflammation and immunity in response to a variety of diseases and infectious processes in the host. Chemokines are a superfamily of homologous 8-10 kDa heparin-binding proteins, originally identified for their role in mediating leukocyte recruitment. The four major families of chemokine ligands are classified on the basis of a conserved amino acid sequence at their amino terminus, and are designated CXC, CC, C, and CX3C sub-families (where “X” is a non-conserved amino acid residue).

The CXC chemokines are one of the largest families of chemokines, and each member of this group contains four cysteine residues. Most chemokines are small proteins (8-10 kDa in size), have a net positive charge at neutral pH, and share considerable amino acid sequence homology. Structurally, the defining feature of the CXC chemokine family is a motif of four conserved cysteine residues, the first two of which are separated by a non-conserved amino acid, thus constituting the Cys-X-Cys or ‘CXC’ motif. This family is further subdivided on the basis of the presence or absence of another three amino acid sequence, glutamic acid-leucine-arginine (the ‘ELR’ motif), immediately proximal to the CXC sequence. The ELR-positive (ELR⁺) CXC chemokines, which include IL-8/CXCL8, are potent neutrophil chemoattractants and promote angiogenesis. Among the ELR-negative (ELR⁻) CXC chemokines, CXCL9, CXCL10, and CXCL11 are potently induced by both type 1 and type 2 interferons (IFN-α/β and IFN-γ, respectively). These Interferon-inducible (ELR⁻) CXC chemokines are generated by a variety of cell types including monocytes, macrophages, lymphocytes, and epithelial cells, and are extremely potent chemoattractants for recruiting mononuclear leukocytes, including activated Th1 CD4 T cells, natural killer (NK) cells, NKT cells, and dendritic cells to sites of inflammation and inhibiting angiogenesis.

The chemokine receptors are a family of related receptors that are expressed on the surface of leukocytes as well as other cells. The shared receptor for CXCL9, CXCL10, and CXCL11 is CXCR3. Through their interaction with CXCR3, the ligands CXCL9, CXCL10, and CXCL11 are the major recruiters of specific leukocytes, including CD4 T cells, NK cells, and myeloid dendritic cells. Importantly, this chemokine ligand-receptor system is at the core of a positive feedback loop escalating Th1 immunity, whereby cytokines such as interleukin (IL)-12 and IL-18 (released by myeloid accessory cells) activate local NK cells to produce IFN-γ, thereby inducing the generation of CXCL9, CXCL10, and CXCL11, which then recruits CXCR3-expressing cells that act as a further source of IFN-γ, which then induces further production of CXCL9, CXCL10, and CXCL11. Consistent with the importance of these interferon-inducible (ELR⁻) CXC chemokines in promoting Th1-mediated immunity, CXCR3 and its ligands have been documented to play a critical role in host defense against many microorganisms, including viruses, Mycobacterium tuberculosis, bacteria, and protozoa.

Independent of their role in CXCR3-dependent leukocyte recruitment, CXCL9, CXCL10, and CXCL11 have also been found to display direct antimicrobial properties that resemble those of defensins. These antimicrobial effects were first demonstrated in 2001 against Escherichia coli and Listeria monocytogenes. Subsequently, an increasing number of chemokines have been shown to have antimicrobial activity against various strains of bacteria and fungi, including Escherichia coli, Staphylococcus aureus, Candida albicans, and Cryptococcus neoformans.

Previously, the present inventors have described chemokine-derived peptides and derivatives and conjugates thereof for use in treating and/or preventing bacterial infections (see United States Patent Application Publication No. 2021/0300981 and PCT International Patent Application Publication No. WO 2020/023670, the disclosure of each of which is incorporated by reference herein in its entirety). These derivatives and conjugates were centered on the base peptide RTVRCTCI and peptides with single or multiple amino acid substitutions as well as conjugates with other peptides (including but not limited to peptides comprising the amino acid sequence LSRTVRCTCISI (SEQ ID NO: 109) or VPLSRTVRCTCISI (SEQ ID NO: 110), optionally linked with a peptide and/or non-peptide linker, D-amino acid derivatives thereof, polymers thereof, etc.

The present subject matter extends that previous work by disclosing that in some embodiments, dimers of the base peptide RTVRCTCI (SEQ ID NO: 1) have enhanced anti-bacterial activity relative to the base peptide. This is particularly the case with dimers of the base peptide RTVRCTCI (SEQ ID NO: 1), wherein each amino acid is a D-amino acid. Furthermore, as disclosed herein, in some embodiments the antiparallel dimers have enhanced anti-bacterial activity relative to parallel dimers.

### II. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the term “D8” refers to an exemplary peptide of the presently disclosed subject matter that has the amino acid sequence RTVRCTCI (SEQ ID NO: 1) in which all eight amino acids are D-amino acids. Similarly, the term “L8” refers to an exemplary peptide of the presently disclosed subject matter that has the amino acid sequence RTVRCTCI (SEQ ID NO: 1) in which all eight amino acids are L-amino acids. For peptides with mixed D-amino acids and L-amino acids, D-amino acids are indicated with a “D” preceding the one letter code for the amino acid. Similarly, L-amino acids are indicated with a “L” preceding the one letter code for the amino acid or, more frequently, are indicated by amino acids that lack either a “D” or an “L” preceding the one letter code for the amino acid. It is noted, however, that amino acids that lack either a “D” or an “L” preceding the one letter code for the amino acid can in some embodiments be either D-amino acids or L-amino acids.

A disease or disorder is “alleviated” if the severity of a symptom of the disease, condition, or disorder, or the frequency with which such a symptom is experienced by a subject, or both, are reduced by any measurable criterion. In some embodiments, a disease or disorder is “alleviated” if the severity of a symptom of the disease, condition, or disorder, or the frequency with which such a symptom is experienced by a subject, or both, are reduced to a condition that would be considered to be normal (i.e., absent).

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and includes humans. In the context of the invention, the term “subject” generally refers to an individual who will receive or who has received treatment for a condition characterized by the presence of bacteria (e.g., Bacillus anthracis in any stage of its growth cycle), or in anticipation of possible exposure to bacteria. As used herein, the terms “subject” and “patient” are used interchangeably, unless otherwise noted.

As used herein, the terms “neutralize” and “neutralization” when used in reference to bacterial cells or spores (e.g., B. anthracis cells and spores) refers to a reduction in the ability of the spores to germinate and/or cells to proliferate.

As used herein the term “bacterial spore” or “spore” is used to refer to any dormant, non-reproductive, but viable structure produced by some bacteria (e.g., Bacillus and Clostridium) in response to adverse environmental conditions.

As used herein, the term “treating a surface” refers to the act of exposing a surface to one or more compositions of the present invention. Methods of treating a surface include, but are not limited to, spraying, misting, submerging, wiping, and coating. Surfaces include organic surfaces (e.g., food products, surfaces of animals, skin, etc.) and inorganic surfaces (e.g., medical devices, countertops, instruments, articles of commerce, clothing, etc.).

As used herein, the terms “effective amount” and “therapeutically effective amount” are used interchangeably and refer to the amount that provides a therapeutic effect, e.g., an amount of a composition that is effective to treat or prevent pathological conditions, including signs and/or symptoms of disease, associated with a pathogenic organism infection (e.g., spore germination, bacterial growth, toxin production, etc.) in a subject.

The terms “bacteria” and “bacterium” refer to all prokaryotic organisms, including those within all of the phyla in the Kingdom Procaryotae. As used herein, the term “microorganism” refers to any species or type of microorganism, including but not limited to, bacteria, archaea, fungi, protozoans, mycoplasma, and parasitic organisms.

As used herein the term “colonization” refers to the presence of bacteria in a subject that are either not found in healthy subjects, or the presence of an abnormal quantity and/or location of bacteria in a subject relative to a healthy patient.

The term “stationary growth phase” as used herein defines the growth characteristics of a given population of microorganisms. During a stationary growth phase, the population of bacteria remains stable with the rate of bacterial division being approximately equal to the rate of bacterial death. This can be due to increased generation time of the bacteria. Accordingly, “stationary phase bacteria” are bacteria that are in a stationary growth phase. “Exponential phase bacteria” are bacteria that are rapidly proliferating, and the population is rapidly expanding, typically the number of bacteria increases at an exponential rate.

As used herein a “multidrug-resistant” (or “MDR”) microorganism or bacteria is an organism that has an enhanced ability, relative to non-resistant strains, to resist distinct drugs or chemicals (of a wide variety of structure and function) targeted at eradicating the organism. Typically, the term refers to resistance to at least 3 classes of antibiotics.

Chemokines are small proteins secreted by cells that have the ability to induce directed chemotaxis in responsive cells. As used herein the term “interferon-inducible (ELR⁻) CXC chemokine” refers to a chemokine protein, or corresponding peptidomimetic, having a motif of four conserved cysteine residues, the first two of which are separated by a non-conserved amino acid (thus constituting the Cys-X-Cys or ‘CXC’ motif) and devoid of a three amino acid sequence, glutamic acid-leucine-arginine (the ‘ELR’ motif), immediately proximal to the CXC sequence. Examples of interferon-inducible (ELR⁻)CXC chemokines include human CXCL9, murine CXCL9, human CXCL10 (SEQ ID NO: 1), murine CXCL10, human CXCL11, and murine CXCL11. CXCL9, CXCL10, and CXCL11 are potently induced by both type 1 and type 2 interferons (IFN-α/β and IFN-γ, respectively).

As used herein, the term “adjuvant” as used herein refers to an agent which enhances the pharmaceutical effect of another agent.

The expression “amino acid” as used herein is meant to include both natural and synthetic amino acids, and both D- and L-amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. In some embodiments, one or more of the amino acids of the peptides, dimers, conjugates, and/or derivatives thereof of the presently disclosed subject matter are D-amino acids, and in some embodiments all of the amino acids of the peptides, dimers, conjugates, and/or derivatives thereof of the presently disclosed subject matter are D-amino acids.

Additionally, one or more disulfide linkage can be present or absent in the peptides of the presently disclosed subject matter. In some embodiments of the dimers, conjugates, and/or derivatives thereof of the presently disclosed subject matter, there are one or two disulfide linkages, which in some embodiments are disulfide linkages between cysteines of different individual peptide monomers to form peptide dimers. Thus, in some embodiments a peptide of the presently disclosed subject matter, optionally a peptide with the amino acid sequence RTVRCTCI (SEQ ID NO: 1) or a derivative thereof, is a cyclized peptide by formation of an intramolecular disulfide bond between two cysteines present in the peptide (including but not limited to the cysteines at positions 5 of 7 of the peptide all-D-RTVRCTCI (SEQ ID NO: 1) or the derivative thereof).

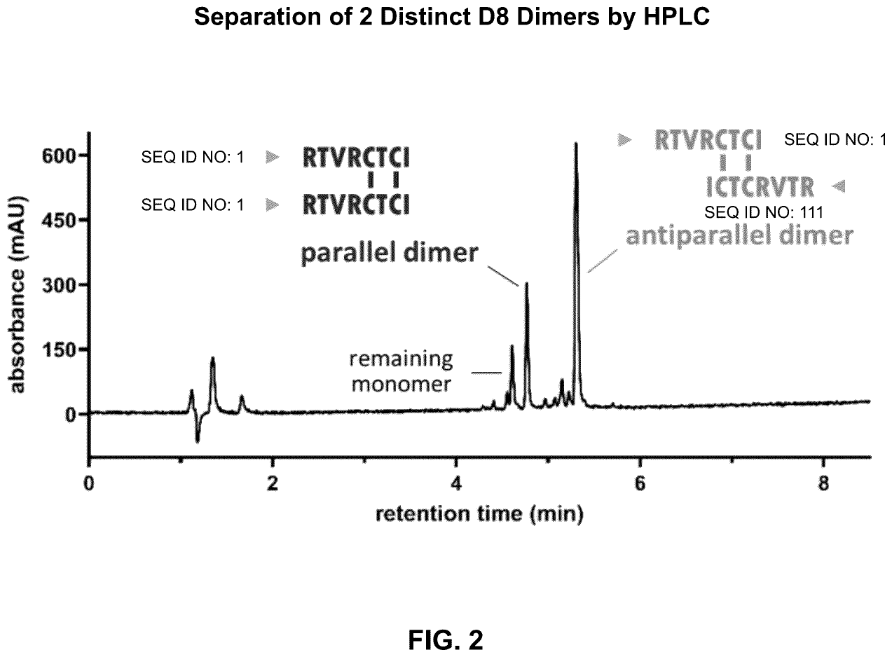

In some embodiments, the peptide of the presently disclosed subject matter, optionally a peptide with the amino acid sequence RTVRCTCI (SEQ ID NO: 1) or a derivative thereof, is a dimer resulting from one or more (including but not limited to in some embodiments 1 and in some embodiments 2) intermolecular disulfide bond between cysteines present in different individual peptides. By way of example and not limitation, peptide dimers can form when two peptides comprising the amino acid sequence RTVRCTCI (SEQ ID NO: 1) or derivatives thereof form one or two intermolecular cysteine disulfide bonds. In those embodiments of two peptides comprising the amino acid sequence RTVRCTCI (SEQ ID NO: 1) or derivatives thereof in which one intermolecular cysteine disulfide bond is formed, that intermolecular disulfide bond can involve cysteine 5 of the first peptide with cysteine 5 or cysteine 7 of the second peptide. However, when two peptides comprising the amino acid sequence RTVRCTCI (SEQ ID NO: 1) or derivatives thereof form a dimer having two intermolecular cysteine disulfide bonds, those intermolecular disulfide bond can involve cysteine 5 of the first peptide with cysteine 5 of the second peptide and thus cysteine 7 of the first peptide with cysteine 7 of the second peptide. This would result in a parallel dimer. On the other hand, when two peptides comprising the amino acid sequence RTVRCTCI (SEQ ID NO: 1) or derivatives thereof form a dimer having two intermolecular cysteine disulfide bonds, those intermolecular disulfide bond can also involve cysteine 5 of the first peptide with cysteine 7 of the second peptide and thus cysteine 7 of the first peptide with cysteine 5 of the second peptide. This would result in an antiparallel dimer. These structures are depicted in FIG. 2. It is to be understood that any dimer described herein can comprise any 2 peptides described herein in either a parallel or anti-parallel configuration.

The term “amino acid” is used interchangeably with “amino acid residue” and can refer to a free amino acid and to an amino acid residue of a peptide. It will be apparent from the context in which the term is used whether it refers to a free amino acid or a residue of a peptide.

The term “antibody”, as used herein, refers to an immunoglobulin molecule which is able to specifically bind to a specific epitope on an antigen. Antibodies can be derived from natural sources or from recombinant sources and can be intact immunoglobulins or immunoreactive portions of intact immunoglobulins (for example, a fragment or derivative of an antibody that includes an antigen-binding site or a paratope). Antibodies are typically tetramers of immunoglobulin molecules. The antibodies in the present invention can exist in a variety of forms including, for example, polyclonal antibodies, monoclonal antibodies, Fv, Fab and F(ab)₂, as well as single chain antibodies and humanized antibodies (see e.g., Bird et al., 1988; Harlow & Lane, 1988; Houston et al., 1988; Harlow & Lane, 1999; each of which is incorporated herein by reference in its entirety).

The term “synthetic antibody” as used herein refers to an antibody which is generated using recombinant DNA technology, such as, for example, an antibody expressed by a bacteriophage or a host cell. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antimicrobial agent”, as used herein, refers to any entity that exhibits antimicrobial activity, i.e. the ability to inhibit the growth of and/or kill bacteria, including for example the ability to inhibit growth or reduce viability of bacteria by at least about 30%, at least about 40%, at least about 50%, at least about 60%, at least about 70% or more than 70%, as compared to bacteria not exposed to the antimicrobial agent. The antimicrobial agent can exert its effect either directly or indirectly and can be selected from a library of diverse compounds, including for example antibiotics. For example, various antimicrobial agents act, inter alia, by interfering with (1) cell wall synthesis, (2) plasma membrane integrity, (3) nucleic acid synthesis, (4) ribosomal function, and (5) folate synthesis. One of ordinary skill in the art will appreciate that a number of“antimicrobial susceptibility” tests can be used to determine the efficacy of a candidate antimicrobial agent.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (see e.g., U.S. Pat. No. 5,034,506 to Summerton & Weller; Nielsen et al., 1991). The term “antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences.

As used herein, the term “biologically active fragments” or “bioactive fragment” of a polypeptide encompasses natural or synthetic portions of the full-length protein that are capable of specific binding to their natural ligand or of performing the function of the protein.

A “pathogenic” cell is a cell which, when present in a tissue, causes or contributes to a disease or disorder in the animal in which the tissue is located (or from which the tissue was obtained).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T”, is complementary to the sequence “T-C-A.”

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, the term “cytokine” refers to an intercellular signaling molecule, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that can be produced from another compound of similar structure in one or more steps, as in replacement of H by an alkyl, acyl, or amino group. Similarly, a “derivative” of a peptide (or of a polypeptide) is a compound that can be produced from or has a biological activity similar to a peptide (or a polypeptide) but that differs in the primary amino acid sequence of the peptide (or the polypeptide) to some degree. By way of example and not limitation, a derivative of a subject peptide of the presently disclosed subject matter is a peptide that has a similar although not identical primary amino acid sequence as the subject peptide (for example, has one or more amino acid substitutions) and/or that has one or more other modifications (e.g., N-terminal, C-terminal, and/or internal modifications) as compared to the subject peptide. Thus, the term “derivative” compasses the term “modified peptide” and vice versa, in the context of peptides. In some embodiments, a derivative of a peptide is a peptide that has one or more moieties conjugated thereto, including but not limited to the modifications exemplified in Table 3 below.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid of the amino acid sequence, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool; Altschul et al., 1990) are available for determining sequence identity.

In some embodiments, “identity” can be expressed as a “percent identity”. As used herein, the phrase “percent identity” in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981; by the homology alignment algorithm disclosed in Needleman & Wunsch, 1970; by the search for similarity method disclosed in Pearson & Lipman, 1988; by computerized implementations of these algorithms (GAP, BESTFIT, FASTA, and TFASTA in the GCG® WISCONSIN PACKAGE®, available from Accelrys, Inc., San Diego, California, United States of America), or by visual inspection. See generally, Altschul et al., 1990; Ausubel et al., 2002; and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

As used herein, the term “inhibit” refers to the ability of a compound or any agent to reduce or impede a described function or pathway. For example, inhibition can be in some embodiments by at least 10%, in some embodiments by at least 25%, in some embodiments by at least 50%, in some embodiments by at least 75%, in some embodiments by at least 80%, in some embodiments by at least 85%, in some embodiments by at least 90%, in some embodiments by at least 95%, in some embodiments by at least 97%, in some embodiments by at least 99%, and in some embodiments by greater than 99%.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound invention or be shipped together with a container which contains the identified compound. Alternatively, the instructional material can be shipped separately from the container with the intention that the instructional material and the compound be used cooperatively by the recipient.

An “isolated” compound/moiety is a compound/moiety that has been removed from components naturally associated with the compound/moiety. For example, an “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “modulate” refers to changing the level of an activity, function, or process. The term “modulate” encompasses both inhibiting and stimulating an activity, function, or process.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides (e.g., a polypeptide of in some embodiments at least 50 amino acids, in some embodiments at least 75 amino acids, in some embodiments at least 100 amino acids, in some embodiments at least 200 amino acids, in some embodiments at least 300 amino acids, in some embodiments at least 500 amino acids, and in some embodiments more than 500 amino acids).

A peptide encompasses a sequence of 2 or more amino acids wherein the amino acids are naturally occurring or synthetic (non-naturally occurring) amino acids.

The term “linked” or like terms refers to a connection between two entities. The linkage can comprise a covalent, ionic, or hydrogen bond or other interaction that binds two compounds or substances to one another.

As used herein the term “peptidomimetic” refers to a chemical compound having a structure that is different from the general structure of an existing peptide, but that functions in a manner similar to the existing peptide, e.g., by mimicking the biological activity of that peptide. The term “modified peptide” encompasses a peptidomimetic. Peptidomimetics typically comprise naturally occurring amino acids and/or unnatural amino acids, but can also comprise modifications to the peptide backbone. For example, a peptidomimetic can include one or more of the following modifications:
1. Peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C₁-C₆ alkyl;
2. Peptides wherein the N-terminus is derivatized to a —NRR1 group, to a —NRC(O)R group, to a —NRC(O)OR group, to a —NRS(O)₂R group, to a —NHC(O)NHR group where R and R1 are hydrogen or C₁-C₆ alkyl with the proviso that R and R1 are not both hydrogen;
3. Peptides wherein the C terminus is derivatized to —C(O)R2 where R2 is selected from the group consisting of C₁-C₆ alkoxy, and —NR3R4 where R3 and R4 are independently selected from the group consisting of hydrogen and C₁-C₄ alkyl;
4. Modification of a sequence of naturally occurring amino acids with the insertion or substitution of a non-peptide moiety, e.g., a retroinverso fragment.

The term “permeability”, as used herein, refers to transit of fluid, cell, or debris between or through cells and tissues.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds”, as used herein, is meant a compound which recognizes and binds a specific protein, but does not substantially recognize or bind other molecules in a sample, or it means binding between two or more proteins as in part of a cellular regulatory process, where said proteins do not substantially recognize or bind other proteins in a sample.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse, and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein an “amino acid modification” refers in some embodiments to a substitution, addition, or deletion of an amino acid, and includes substitution with, or addition of, any of the 20 amino acids commonly found in human proteins, as well as unusual or non-naturally occurring amino acids such as but not limited to D-amino acids. Commercial sources of unusual amino acids include Sigma-Aldrich (Milwaukee, Wisconsin, United States of America), ChemPep Inc. (Miami, Florida, United States of America), and Genzyme Pharmaceuticals (Cambridge, Massachusetts, United States of America). Unusual amino acids can be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid. The term “modified peptide” encompasses any amino acid modification as described herein.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substitutions can be designed based on, for example, the model of Dayhoff et al., 1978.

In some embodiments, an amino acid substitution is a conservative amino acid substitution. As used herein, the term “conservative amino acid substitution” is defined in some embodiments as exchanges within one of the following five groups:
I. Small aliphatic, nonpolar, or slightly polar residues: Ala, Ser, Thr, Pro, Gly;
II. Polar, charged residues and their amides: Asp, Asn, Glu, Gln, His, Arg, Lys;
III. Large, aliphatic, nonpolar residues: Met Leu, Ile, Val, Cys
IV. Large, aromatic residues: Phe, Tyr, Trp

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., 1990.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet, or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. By way of example and not limitation, the following substitutions can be made: arginine and lysine; glutamate and aspartate; serine and threonine; glutamine and asparagine; and valine, leucine, and isoleucine. Alternatively, Table 2 lists exemplary conservative amino acid substitutions.

TABLE 2
Exemplary Conservative Amino Acid Substitutions

| Amino Acid | Possible Substitution(s) |
|---|---|
| Ala (A) | Leu, Ile, Val |
| Arg (R) | Gln, Asn, Lys |
| Asn (N) | His, Asp, Lys, Arg, Gln |
| Asp (D) | Asn, Glu |
| Cys (C) | Ala, Ser |
| Gln (Q) | Glu, Asn |
| Glu (E) | Gln, Asp |
| Gly (G) | Ala |
| His (H) | Asn, Gln, Lys, Arg |
| Ile (I) | Val, Met, Ala, Phe, Leu |
| Leu (L) | Val, Met, Ala, Phe, Ile |
| Lys (K) | Gln, Asn, Arg |
| Met (M) | Phe, Ile, Leu |
| Phe (F) | Leu, Val, Ile, Ala, Tyr |
| Pro (P) | Ala |
| Ser (S) | Thr |
| Thr (T) | Ser |
| Trp (W) | Phe, Tyr |
| Tyr (Y) | Trp, Phe, Thr, Ser |
| Val (V) | Ile, Leu, Met, Phe, Ala |

In some embodiments, another consideration for amino acid substitutions include whether or not the residue is located in the interior of a protein or is solvent exposed. For interior residues, conservative substitutions can include in some embodiments: Asp and Asn; Ser and Thr; Ser and Ala; Thr and Ala; Ala and Gly; Ile and Val; Val and Leu; Leu and Ile; Leu and Met; Phe and Tyr; Tyr and Trp. For solvent exposed residues, conservative substitutions can include in some embodiments: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, the Dayhoff matrix, the Grantham matrix, the McLachlan matrix, the Doolittle matrix, the Henikoff matrix, the Miyata matrix, the Fitch matrix, the Jones matrix, the Rao matrix, the Levin matrix, and the Risler matrix (summarized in, for example, Johnson & Overington, 1993; see also the PROWL resource available at the website of The Rockefeller University, New York, New York, United States of America).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### III. Compositions

### III.A. Peptides, Modified Peptides, and Dimers Thereof

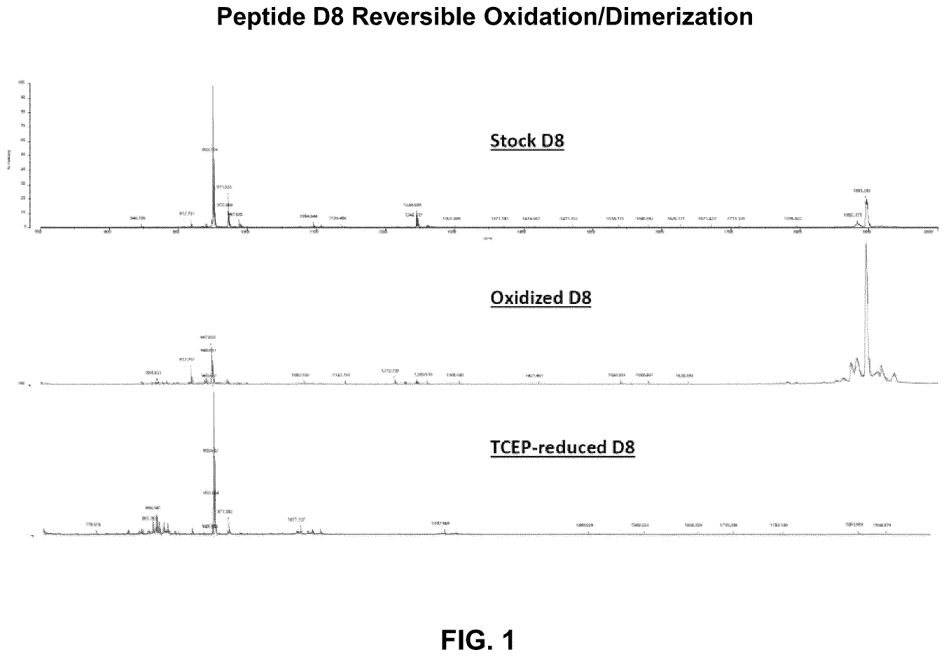

In some embodiments, the presently disclosed subject matter relates to dimers of peptides and/or modified peptides, and combinations thereof, that have antimicrobial activity. The peptides that are employed to produce the peptide dimers of the presently disclosed subject matter were initially identified by consideration of the structure of the human CXCL10 (hCXCL10) gene product. CXCL10 is a small, 10 kiloDalton (kDa) protein that is produced by a variety of cells. The basic structure of hCXCL10 polypeptide is depicted in FIG. 1 of U.S. Provisional Patent Application Ser. No. 63/713,000. In certain aspects, peptides described herein, without intending to be limiting, can be related to one or more of the following IDs relating to CXCL10 and publications describing such: PMCID: PMC9841529; PMID: 11441062; PMCID: PMC5483895; PMID: 12571234; and PMID: 12173928.

As shown in FIG. 1 of U.S. Provisional Patent Application Ser. No. 63/713,000, hCXCL10 is characterized by several domains, including an N-terminus that is involved with interacting with the CXCR3 receptor to regulate chemotaxis and other immunomodulatory effects and a C-terminus that broadly resembles antimicrobial peptides in that it comprises a cationic amphipathic helix structure. However, it has been determined that the N-terminus also has unexpected antimicrobial activity. Between the N-terminus and the C-terminus there are three β sheets as well as the CXC domain, the two cysteines of which create cysteine-cysteine disulfide bridges, the first with a cysteine present between the first and second β sheets and the second with a cysteine present just N-terminal to the α helix present in the C-terminal domain. hCXC10 itself has potent activity in recruiting immune cells to sites of inflammation by virtue of its N-terminal domain interacting with CXCR3, as well as other bioregulatory activities. Full-length hCXCL10 also has direct antimicrobial activity against a variety of pathogens such as but not limited to Bacillus anthracis, Acinetobacter baumannii, and New Delhi metallo-β-lactamase (NDM) producing Klebsiella pneumoniae.

As disclosed herein, dimers of peptides derived from the N-terminus of hCXCL10 have been identified as having antimicrobial activity, including bactericidal activity. Thus, in some embodiments the presently disclosed subject matter relates to dimers of peptides comprising, consisting essentially of, or consisting of the amino acid sequence RTVRCTCI (SEQ ID NO: 1), or peptides with modified amino acid sequences thereof. Exemplary such peptides include peptides comprising, consisting essentially of, or consisting of the amino acid sequences LSRTVRCTCISI (SEQ ID NO: 109) and VPLSRTVRCTCISI (SEQ ID NO: 110), or modified peptides thereof.

As used herein, a “modified peptide” is a peptide that in some embodiments includes one or more amino acid substitutions relative to the base sequence RTVRCTCI (SEQ ID NO: 1). Such peptides can comprise, consist essentially of, or consist of the amino acid sequence RFVRCTCI (SEQ ID NO: 14), RWVRCTCI (SEQ ID NO: 18), RYVRCTCI (SEQ ID NO: 19), RTVRCRCI (SEQ ID NO: 26), RTVRCMCI (SEQ ID NO: 37), RYVRCRCI (SEQ ID NO: 59), RRVRCRCI (SEQ ID NO: 85), and/or RWVRCWCI (SEQ ID NO: 108). It is important to note, however, that in order to dimerize via disulfide linkages, a modified peptide of the presently disclosed subject matter should comprise one or more cysteines. In certain aspects, dimers described herein are joined to each other by one or more disulfide bonds. In other aspects, a peptide described herein is dimerized with another by a chemical linkage other disulfide linkage. As such and with reference to the amino acid sequences presented herein above, if either or both of cysteines 5 and 7 are substituted, a further substitution to “reintroduce” one or more cysteines should also be present. As such, in some embodiments a modified peptide of the presently disclosed subject matter includes one or more amino acid substitutions with the proviso that the two peptides that generate the dimer should both have at least one and optionally two or more cysteines.

Additionally, a modified peptide of the presently disclosed subject matter is characterized in some embodiments by one or more and optionally all of the amino acids being D-amino acids. With particular reference to exemplary peptide RTVRCTCI (SEQ ID NO: 1), when all of the amino acids are D-amino acids, the peptide is referred to herein as all-D-RTVRCTCI (SEQ ID NO: 1) or, more simply, D8.

Thus, in some embodiments, one, two, three, four, five, six, seven, eight, nine, ten, or more amino acids of a peptide of the presently disclosed subject matter are D-amino acids, and in some embodiments all of the amino acids of a peptide of the presently disclosed subject matter are D-amino acids. Acceptable amino acid substitutions are those that do not negatively affect the anti-bacterial ability of the D-amino acid-containing peptide. A peptide having an identical amino acid sequence to that found within a parent peptide but in which all L-amino acids have been substituted with all D-amino acids is also referred to as an “inverso” compounds. For example, if a parent peptide is Arg-Thr-Val, the inverso form is D-Arg-D-Thr-D-Val. By way of example and not limitation, the amino acid sequence RTVRCTCI (SEQ ID NO: 1) where all eight amino acids are L-amino acids is referred to herein as Peptide L8 or just L8. When each amino acid of the amino acids in the amino acid sequence RTVRCTCI (SEQ ID NO: 1) is a D-amino acid, the peptide is the inverso form of Peptide L8, and is referred to herein as Peptide D8 or just D8.

For Peptide D8, a replacement scan (examples of which are shown in FIG. 2 of U.S. Provisional Patent Application Ser. No. 63/713,000) substituted amino acid positions 1, 3, 4, 5, 7 or 8 with other D-amino acids (alanine or serine). Characterization of specific substitutions at other positions and in other peptides provides substitutions, or combinations thereof, that enhance peptide activity or advantageous pharmaceutical properties (e.g. solubility, stability, etc.).

In some embodiments, a peptide of the presently disclosed subject matter is a modified peptide that comprises a modification at the N-terminus, the C-terminus, or both the N-terminus and the C-terminus. In some embodiments, wherein the N-terminal and/or the C-terminal modification is selected from the group consisting of an addition or an aminohexanoic acid (AHX), an azido alanine (Ala(N₃)), a benzoylbenzoic acid (4-BBA), an azido phenylalanine (Phe(4-N₃)), a propargylglycine (PRA), or diazirine-containing amino acid residues, and/or is a replacement of a phenylalanine with an azido phenylalanine (Phe(4-N₃)). In some embodiments, the modification at the N-terminus and/or the C-terminus comprises insertion of a chemically-modified and/or unnatural amino acid at or near (e.g., within 1, 2, 3, 4, 5, or 6 amino acids) of the N- and/or C-terminus of a peptide of the presently disclosed subject matter.

In some embodiments, the peptides of the presently disclosed subject matter have bactericidal and/or bacteriostatic activity against various bacteria. Exemplary bacteria for which the presently disclosed peptides have antibacterial activity include, but are not limited to Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to serovars of Salmonella enterica, including but not limited to Salmonella enterica serovar Typhi, and Shigella flexneri; and biothreat agents such as but not limited to Bacillus anthracis in both vegetative and spore forms. In some embodiments, the peptides of the presently disclosed subject matter have antibacterial activity against a multidrug-resistant (MDR) strain of a given bacterium. Exemplary non-limiting MDR bacteria for which the presently disclosed peptides have antibacterial activity include MDR strains of Enterococcus faecium, Staphylococcus aureus, Klebsiella pneumoniae, Acinetobacter baumannii, Pseudomonas aeruginosa, Salmonella enterica, optionally MDR serovars of Salmonella enterica such as but not limited to Salmonella enterica serovar Typhi, and Shigella flexneri.

The presently disclosed subject matter also provides in some embodiments conjugates and polymers comprising one or more of the peptide dimers of the presently disclosed subject matter conjugated to a second active agent. In some embodiments, the second active agent is itself a peptide of the presently disclosed subject matter. By way of example and not limitation, a conjugate peptide dimer of the presently disclosed subject matter can comprise, consist essentially of, or consist of conjugates between the dimers of the amino acid sequence RTVRCTCI (SEQ ID NO: 1), RFVRCTCI (SEQ ID NO: 14), RWVRCTCI (SEQ ID NO: 18), RYVRCTCI (SEQ ID NO: 19), RTVRCRCI (SEQ ID NO: 26), RTVRCMCI (SEQ ID NO: 37), RYVRCRCI (SEQ ID NO: 59), or RRVRCRCI (SEQ ID NO: 85), or modified peptides thereof, or combinations thereof.

In some embodiments, a first peptide can be directly conjugated to a second peptide or the first and second peptides are indirectly conjugated to each other via a linker. In some embodiments, the linker is a peptide linker, optionally a peptide of 1-9 amino acids, further optionally wherein the 1-9 amino acids are each individually selected from the group consisting of glycine and serine. Such conjugates can then be dimerized by forming a disulfide bond between a cysteine present in one conjugate and either a second conjugate or a non-conjugated peptide as disclosed herein. It is understood and dimers of conjugates can thus be dimers of identical peptides or different peptides, including but not limited to peptides of different lengths.

In some embodiments, a peptide dimer of the presently disclosed subject matter includes a modified peptide, wherein the modification is present at the N-terminus, the C-terminus, or both of either one or both of the peptides. By way of example and not limitation, pegylation is possible at either terminus, or even by incorporating non-natural amino acids with PEGylated side chains (such non-natural amino acids incorporated into peptides).

In some embodiments, other modifications are provided. By way of example and not limitation, dyes are incorporated on peptide side chains or at the chain end, such as to facilitate pharmacokinetic studies. Such dyes range from rhodamine B (red) and fluorescein (green) to near-IR dyes that are compatible with animal imaging. Alternatively or in addition, peptide side chains, chain ends, and/or other sites can be modified to comprise one or more moieties that can be detected by imaging techniques including but not limited to photoacoustic imaging (PAI) and/or Positron Emission Tomography (PET), such as but not limited to for the purpose of tracking a peptide in vivo. In some embodiments, an isotopically-labeled amino acid residue can be incorporated into an individual amino acid or amino acid chain for PET scanning, including but not limited to ⁶⁴Cu, ¹²⁴I, ^{76/77}Br, ⁸⁶Y, ⁸⁹Zr, ⁶⁸Ga, ¹⁸F, ¹¹C, ¹²⁵I, ¹²⁴I, ¹³¹I, ¹²³I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ¹⁷⁷Lu, ⁹⁹Tc, and ¹⁵³Sm.

In some embodiments, a dimer of the presently disclosed subject matter is polymer-functionalized, encapsulated in a particle, embedded in and/or on a solid support, impregnated on a dressing, and/or is formulated for use in a nebulizer, for topical administration, and/or for systemic administration. In some embodiments, the peptide dimer that is embedded/immobilized in and/or on a solid support such as a surface of a medical device such as a stent and/or impregnated on a dressing can be released from the solid support and/or the dressing, optionally wherein the release occurs when the solid support and/or the dressing comes in contact with a subject, optionally a bodily fluid, cell, tissue, or organ of a subject. In some embodiments, the release occurs over a pre-determined time frame. In some embodiments, the solid support and/or the dressing comprises a plurality of particles, wherein each particle is associated with, conjugated to, and/or encapsulates a peptide dimer of the presently disclosed subject matter. In some embodiments, the plurality of particles are characterized by different release profiles, at least one of which is a slow-release profile, at least one of which is a fast-release profile, or combinations thereof (see e.g., U.S. Patent Application Publication No. 2011/0218140 for examples of slow-release and fast-release nanoparticles.

Thus, in some embodiments the presently disclosed subject matter relates to a wound dressing, wherein the wound dressing comprises a support layer with an antibacterial agent embedded therein or associated therewith. In some embodiments, the antibacterial agent comprises, consists essentially of, or consists of a peptide dimer as disclosed herein, a conjugate as disclosed herein, a polymer as disclosed herein, or any combination thereof. In some embodiments, the peptide dimer is encapsulated in one or more particles that are embedded in or associated with the support layer. In some embodiments, some or all of the one or more particles are designed to release from the support layer when the dressing is in contact with a wound, and in some embodiments some or all of the one or more particles are designed to be retained in the support layer when the dressing is in contact with a wound.

### III.B. Pharmaceutical Compositions

In some embodiments, the peptide dimers of the presently disclosed subject matter are present in a pharmaceutical composition. Thus, in some embodiments the presently disclosed subject matter relates to pharmaceutical compositions comprising, consisting essentially of, or consisting of one or more peptide dimers as disclosed herein, including dimers of conjugates as disclosed herein, one or more polymers as disclosed herein, or any combination thereof, along with one or more pharmaceutically acceptable carriers, diluents, or excipients. In some embodiments, the presently disclosed pharmaceutical compositions are pharmaceutically acceptable for use in humans.

Thus, the disclosed pharmaceutical compositions can be employed by administration to a subject in need thereof. In some embodiments, the disclosed pharmaceutical compositions can be administered in vivo in a pharmaceutically acceptable carrier. By “pharmaceutically acceptable” is meant a material that is not biologically or otherwise undesirable, i.e., the material can be administered to a subject, along with a peptide dimer composition of the presently disclosed subject matter, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution and/or in suspension (for example, incorporated into microparticles, liposomes, and/or cells.

As would be understood by those of skill in the art, when peptides are synthesized for use in dimer formation, they are typically prepared with counter-ions to stabilize basic/acidic sidechains of amino acid residues. Thus, peptide drugs often occur in the form of salts where different counter-ions can affect peptide structure, function, etc. differently. In particular, cationic peptides like those disclosed herein are typically obtained as trifluoroacetic acid (TFA) salts. However, TFA can be toxic and have off-target effects making it an unfavorable drug formulation for some uses. To address these issues, in some embodiments TFA has been exchanged with acetate or formate, each of which is non-toxic and can be used in peptide pharmaceuticals. In some embodiments, the acetate formulation of a peptide of the presently disclosed subject matter provides better bactericidal activity than the formate formulation.

The peptide dimer, conjugate, and polymer compositions of the presently disclosed subject matter can be used therapeutically in combination with one or more pharmaceutically acceptable carriers.

Suitable carriers and their formulations are described in Remington et al., 1975. Typically, an appropriate amount of a pharmaceutically acceptable salt is used in the formulation to render the formulation isotonic. Examples of the pharmaceutically acceptable carrier include, but are not limited to, saline, Ringer's solution, and dextrose solution. The pH of the solution is in some embodiments from about 5 to about 8, and in some embodiments from about 7 to about 7.5. Further carriers include sustained release preparations such as semipermeable matrices of solid hydrophobic polymers containing the peptide compositions, which matrices are in the form of shaped articles, e.g., films, liposomes, or microparticles. It will be apparent to those persons skilled in the art that certain carriers can be selected depending upon, for instance, the route of administration and/or concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents, and the like, in addition to the molecule of choice. Pharmaceutical compositions can also include one or more active ingredients such as antimicrobial agents, anti-inflammatory agents, anesthetics, and the like.

The pharmaceutical composition can be administered in a number of ways depending on whether local or systemic treatment is desired, and on the area to be treated. Administration can occur topically (including ophthalmically, vaginally, rectally, intranasally), orally, by inhalation, or parenterally, for example by intravenous drip, subcutaneous, intraperitoneal, or intramuscular injection. The disclosed peptide compositions can be administered in some embodiments topically, intravenously, intraperitoneally, intramuscularly, subcutaneously, intracavity, or transdermally.

Preparations for parenteral administration can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like.

Formulations for topical administration can include ointments, lotions, creams, gels, drops, suppositories, sprays, liquids, and powders. Conventional pharmaceutical carriers, aqueous, powder or oily bases, thickeners, and the like can also be employed, as desired.

Compositions for oral administration include powders or granules, suspensions or solutions in water or non-aqueous media, capsules, sachets, or tablets. Thickeners, flavorings, diluents, emulsifiers, dispersing aids, or binders can in some embodiments also be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, or phosphoric acid and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl, and aryl amines and substituted ethanolamines.

### IV. Methods for Using Dimers of the Peptides, Modified Peptides, and Conjugates of the Presently Disclosed Subject Matter

The presently disclosed subject matter compositions and pharmaceutical compositions can be employed for preventing and/or treating microorganismal infections either in vivo, ex vivo, or in vitro. Thus, in some embodiments the presently disclosed subject matter relates to methods for inhibiting the growth of and/or killing a bacterium. In some embodiments, the methods comprise contacting the bacterium with an effective amount of an antibacterial agent, wherein the antibacterial agent comprises, consists essentially of, or consists of one or more peptide dimers as disclosed herein, or any combination thereof.

Infection with both Gram-negative and Gram-positive bacteria can be treated and/or prevented using the compositions and methods of the presently disclosed subject matter. By way of example and not limitation, in some embodiments the bacterium is selected from the group consisting of Enterococcus faecium, Staphylococcus aureus, Klebsiella pneumoniae, Acinetobacter baumannii, Pseudomonas aeruginosa, members of the family Enterobacteriaceae, including but not limited to Escherichia coli, Klebsiella spp., and Enterobacter cloacae; sexually-transmitted bacteria such as but not limited to Neisseria gonorrhoeae; enteric pathogens such as but not limited to serovars of Salmonella enterica, including but not limited to Salmonella enterica serovar Typhi, and Shigella flexneri; and biothreat agents such as but not limited to Bacillus anthracis in both vegetative and spore forms. In some embodiments, the bacterium is selected from the group consisting of Enterococcus spp. including but not limited to Enterococcus faecium such as but not limited to vancomycin-resistant E. faecium (VRE), Staphylococcus aureus including but not limited to methicillin-resistant S. aureus (MRSA), Klebsiella pneumoniae including but not limited to multidrug resistant and carbapenem-resistant K. pneumoniae, Acinetobacter spp. including but not limited to multidrug-resistant Acinetobacter spp., Pseudomonas aeruginosa including but not limited to multidrug-resistant P. aeruginosa, Enterobacteriaceae including but not limited to MDR and/or CRE Escherichia coli and Klebsiella spp., enteric pathogens including but not limited to multidrug-resistant Salmonella enterica serovars such as serovar Typhi and multidrug-resistant Shigella flexneri, sexually-transmitted bacteria such as but not limited to Neisseria gonorrhoeae, and biothreat agents such as but not limited to both the vegetative and spore forms of Bacillus anthracis.

In some embodiments, the presently disclosed subject matter also relates to methods for treating bacterial infections present in wounds. In some embodiments, the methods comprise contacting the wound with an effective amount of a composition comprising one or more peptide dimers as disclosed herein.

In some embodiments, an infection can be a pulmonary infection, and thus the presently disclosed subject matter related in some embodiments to methods for treating pulmonary infections in subjects by administering to a subject in need thereof an effective amount of a composition comprising one or more peptide dimers as disclosed herein. In some embodiments, the composition is administered to the subject intranasally, by inhalation, optionally wherein the one or more peptides in the composition is/are aerosolized, or any combination thereof.

The presently disclosed compositions can also be employed for treating or preventing systemic bacterial infections in subjects. In some embodiments, the methods comprise administering to a subject in need thereof an effective amount of a composition comprising one or more peptide dimers. The presently disclosed compositions can also be employed in a combination therapy in which the composition comprising one or more peptide dimers comprising, consisting essentially of, or consisting of an amino acid sequence as set forth herein, or modified variants thereof, is administered to the subject before, after, or concurrently with a second antibacterial therapy, which in some embodiments can involve the use of a conventional antibiotic. In some embodiments, the conventional antibiotic is selected from the group consisting of penicillins, cephalosporins, carbepenems, other beta-lactams antibiotics, aminoglycosides, macrolides, lincosamides, glycopeptides, tetracylines, chloramphenicol, quinolones, fucidins, sulfonamides, triinethoprims, rifamycins, oxalines, streptogramins, lipopeptides, ketolides, polyenes, azoles, and echinocandins.

Thus, in some embodiments the presently disclosed subject matter relates to any and all uses of the peptide dimers disclosed herein, or any combination thereof for preventing or treating a bacterial infection.

Given that antibacterial activity of the presently disclosed subject matter peptide dimers, the presently disclosed subject matter also provides in some embodiments methods for treating or preventing community and/or nosocomial infections in subjects. In some embodiments, the methods comprise administering to a subject at risk for developing and/or who has developed a community and/or nosocomial infection a composition of the presently disclosed subject matter. In some embodiments, the composition comprises a peptide dimer comprising, consisting essentially of, or consisting of peptides wherein at least one such peptide has an amino acid sequence as set forth herein, a modified peptide thereof, a fragment thereof, or a combination thereof.

Similarly, the presently disclosed subject matter also provides in some embodiments methods for inducing a subject's immune system against a pathogen by administering to the subject a composition comprising a peptide dimer comprising, consisting essentially of, or consisting of an amino acid sequence as set forth herein, or a combination thereof.

### V. Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1

### Peptide D8 Reversible Oxidation/Dimerization

Cysteine residues are able to form disulfide bonds via oxidation reactions. To determine whether the oxidation of peptide D8 results in the formation of peptide multimers, D8 was oxidized and then treated+/−Tris(2-carboxyethyl)phosphine (TCEP), a strong reducing agent capable of converting disulfide bonds back to free thiol groups. All peptide samples were subsequently analyzed by mass spectrometry. As shown in FIG. 1 monomeric peptide D8 (top panel) displayed its expected monomeric mass at ~950 Da. Oxidized peptide D8 (middle panel) displayed twice the mass, consistent with the formation of a dimer (~1,895 Da). After TCEP treatment (bottom panel), oxidized/dimeric peptide D8 was fully reduced back to its monomer form.

### Separation of 2 Distinct D8 Dimers by HPLC

Depending on the arrangement of peptide subunits in a D8 dimer, two orientations are possible: anti-parallel and parallel. Indeed, using unguided oxidation, two separable products can be generated from monomeric D8 and separated using high-performance liquid chromatography (HPLC). Subsequently, we prepared each possible dimer orientation via direct synthesis to assign peak 1 (parallel) and peak 2 (anti-parallel), as shown in FIG. 2.

### Mass Spectra of Anti-Parallel and Parallel Dimers

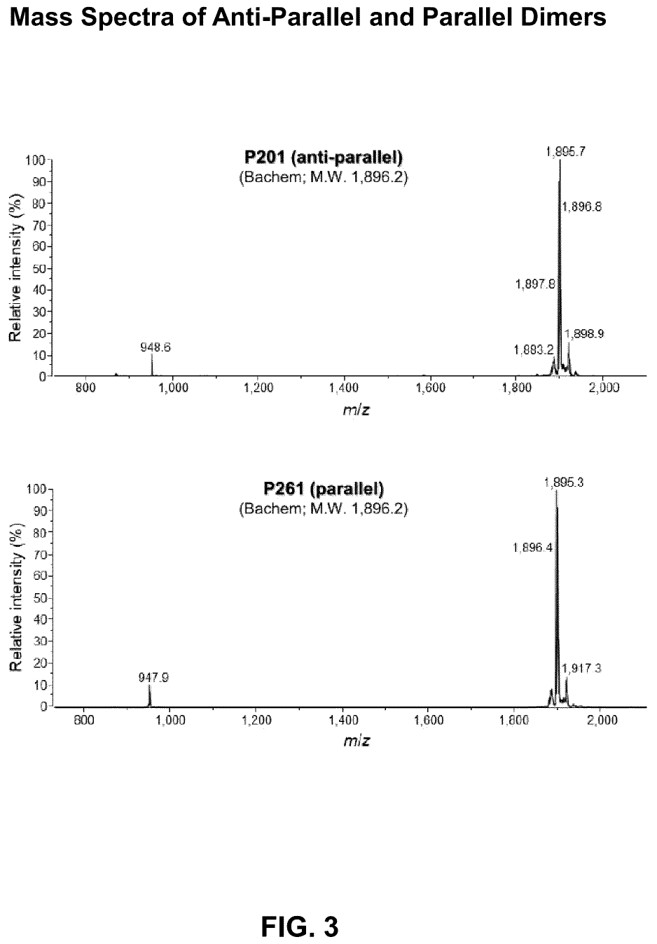

To validate that the two prepared anti-parallel (P201) and parallel (P261) D8 products were dimers, mass spectrometry was used. As shown in FIG. 3, both the anti-parallel and parallel D8 dimers displayed the correct mass (~1,895 Da), thus confirming the successful synthesis of anti-parallel and parallel dimeric forms of D8.

MICs Against Klebsiella pneumoniae Strain 43816

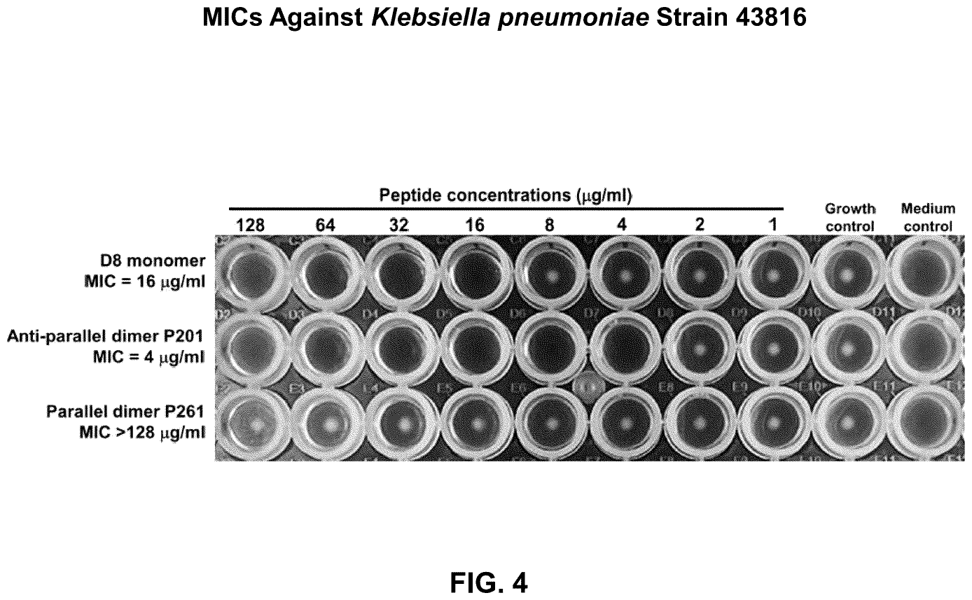

To assess the antimicrobial efficacy of the two dimeric forms of peptide D8, the minimum inhibitory concentrations (MICs) of each were determined against Klebsiella pneumoniae strain ATCC 43816 using the broth microdilution method. As shown in the representative image presented in FIG. 4, the anti-parallel dimer (P201) demonstrated increased antimicrobial activity as compared to the D8 monomer, with an MIC of 4 μg/ml (approximately 4-fold lower than the monomeric control MIC at 16 μg/ml). In contrast, the parallel dimer (P261) showed a significant reduction in activity, with an MIC greater than 128 μg/ml.

Bactericidal Effects Against Klebsiella pneumoniae Over Time

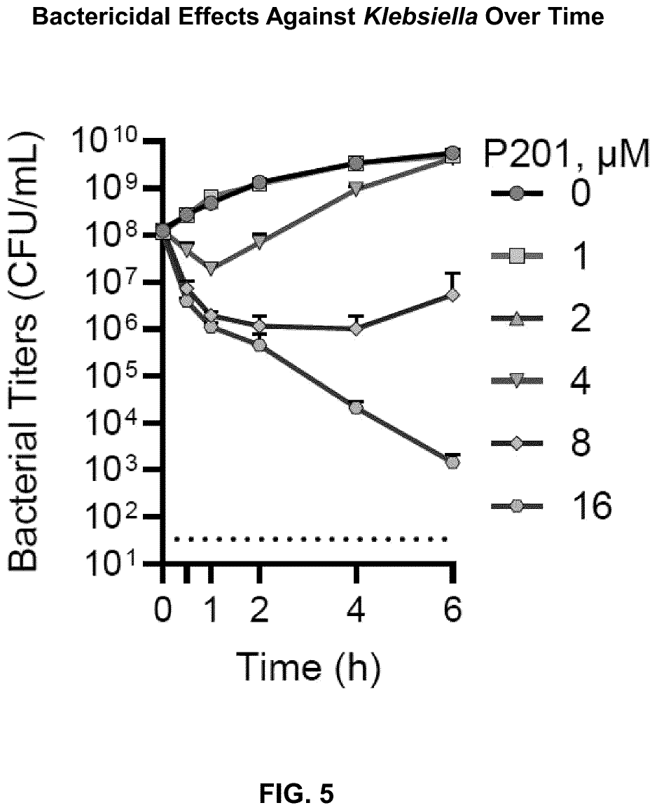

To further evaluate the antimicrobial activity of the anti-parallel dimer (P201), K. pneumoniae strain ATCC 43816 was treated with various concentrations of P201, and bacterial colony-forming unit (cfu) counts were determined at various time points post-exposure. As shown in FIG. 5, P201 exerted bactericidal effects at 4 μM, but at this concentration of peptide, the bacterial culture was able to recover. Increasing amounts of P201 exerted greater bactericidal effects: at 8 and 16 μM P201, bacterial counts were reduced by more than 100-fold within 2 hours of peptide exposure.

Bactericidal Effects Against Klebsiella pneumoniae in Various Media

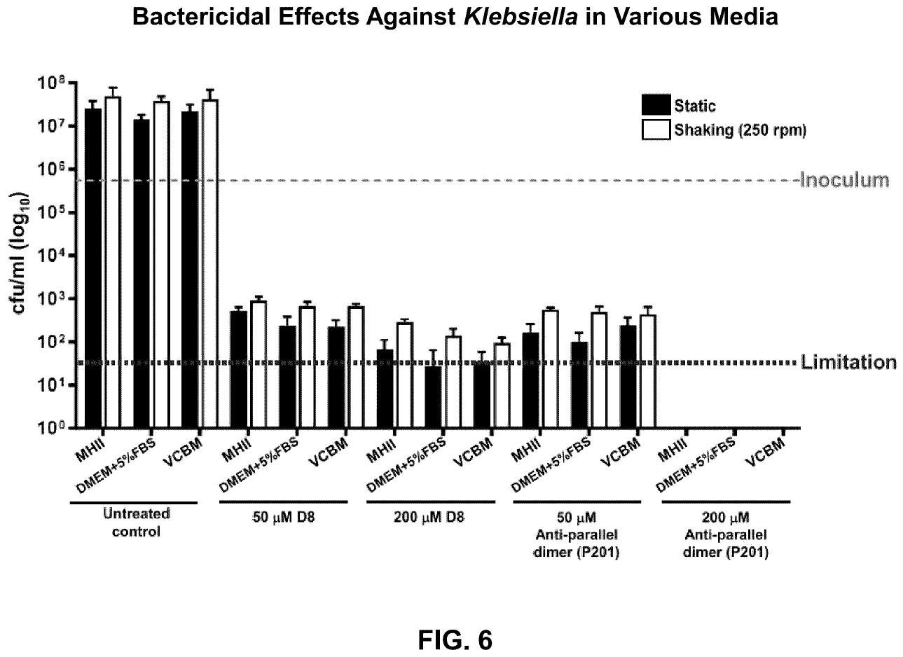

Since medium and/or culture conditions can affect peptide activity and/or bacterial susceptibility, we examined the potential effects of different culture media on the bactericidal efficacy of the anti-parallel dimer (P201). K. pneumoniae strain ATCC 43816 was treated with peptide in two tissue culture media: DMEM supplemented with 5% FBS (DMEM+5% FBS) and Vascular Cell Basal Medium (VCBM). Both are commonly used for culturing immortalized and primary cell lines. Bacteriologic medium, MHII, was used as a control. Two concentrations of P201 (50 and 200 μM) were tested under both static and shaking (250 rpm) conditions. Monomeric peptide D8 served as a control. Bacterial cfu counts were enumerated 2 h post-exposure. As shown in FIG. 6, the bactericidal efficacy of the anti-parallel dimer was unaffected in either DMEM+5% FBS or VCBM. Efficient killing was also observed under either shaking or static conditions. At 200 μM, P201 reduced bacterial titers below the limit of detection.

MICs Against Acinetobacter baumannii Strain 0088

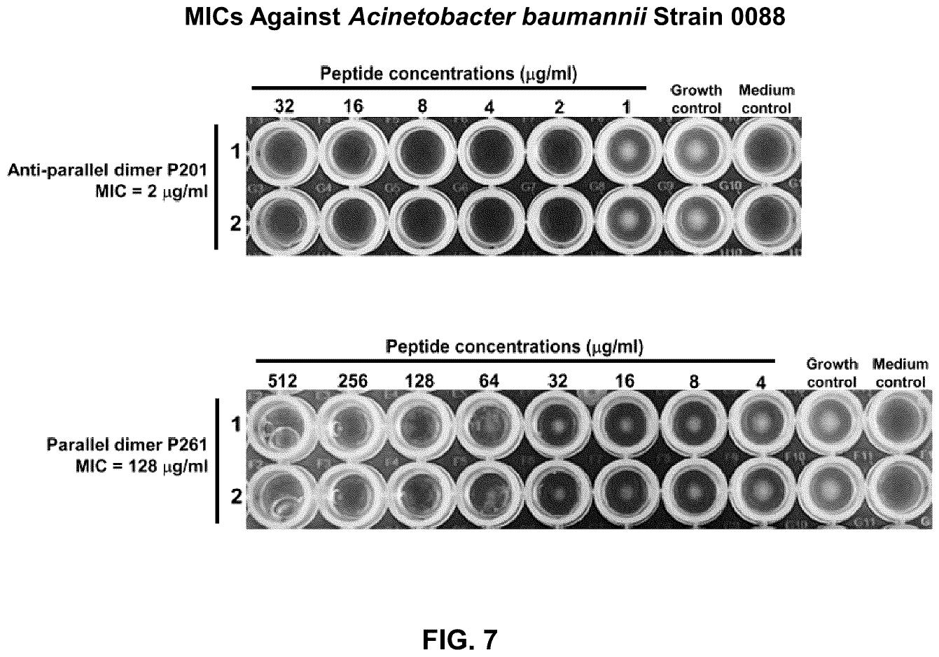

Acinetobacter baumannii is a challenging cause of human infection in healthcare settings, particularly among critically-ill or immunocompromised patients. Moreover, this pathogen is known for its high rates of antibiotic resistance that complicate treatment. To investigate the bactericidal efficacy of the anti-parallel dimer (P201) and the parallel dimer (P261) against this organism, A. baumannii strain 0088 (CDC AR Bank) was used as a reference multidrug-resistant strain for MIC testing. As displayed in FIG. 7, the anti-parallel dimer demonstrated potent antimicrobial efficacy, with an MIC of 2 μg/ml. The parallel dimer P261 exhibited significantly less activity, with an MIC of 128 μg/ml.

MICs Against Acinetobacter baumannii Strain 0282

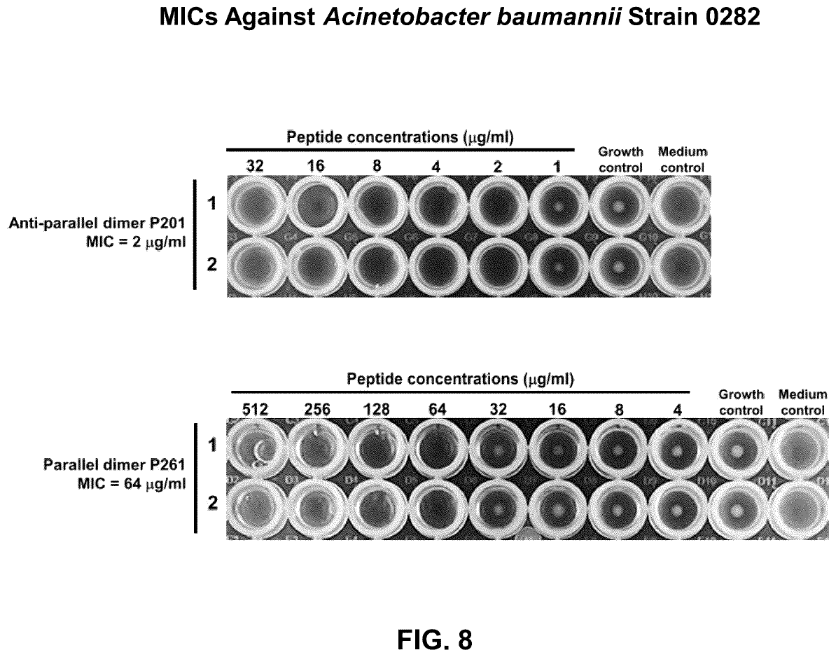

To further assess the bactericidal efficacy of the anti-parallel dimer (P201) and the parallel dimer (P261) against multidrug-resistant (MDR) A. baumannii, strain 0282 (CDC AR Bank) was tested in the broth microdilution assay. As shown in the representative assay plate presented in FIG. 8, the anti-parallel dimer demonstrated effective antimicrobial efficacy, with an MIC of 2 μg/ml, whereas the parallel dimer exhibited reduced activity, with an MIC of 64 μg/ml. These findings indicate that the anti-parallel dimer exerts consistent antimicrobial activity against representative MDR A. baumannii strains.

### MICs Against a Panel of Wild-Type and Antibiotic-Resistant Bacteria

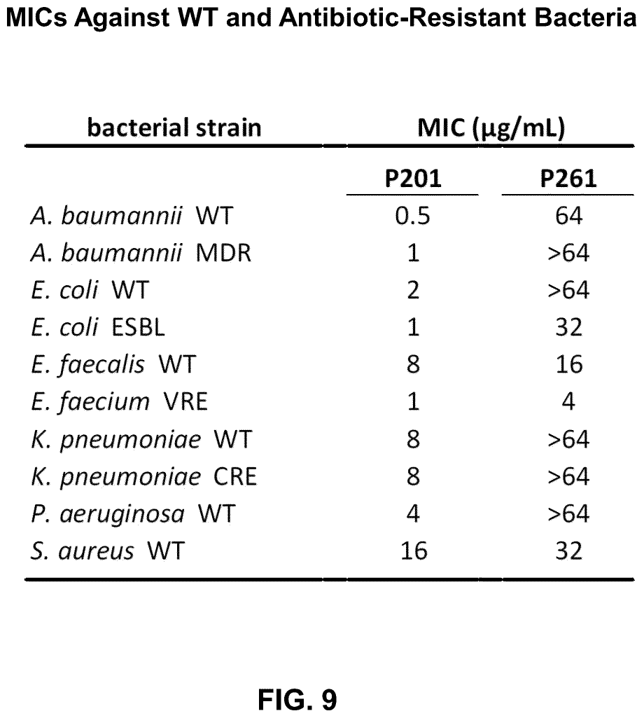

To assess the antimicrobial activity of the anti-parallel dimer (P201) and the parallel dimer (P261) against additional wild-type (WT) and antibiotic-resistant bacteria, the MIC of each dimer was determined against a panel of bacterial strains/isolates: A. baumannii 1208060 (WT), A. baumannii 1215088 (MDR), E. coli 1248389 (extended-spectrum β-lactamase; ESBL), E. coli ATCC 25922 (WT), E. faecalis ATCC 29212 (WT), E. faecium 1208412 (vancomycin-resistant Enterococcus; VRE), K. pneumoniae ATCC 43816 (WT), K. pneumoniae ATCC BAA 1705 (carbapenem-resistant Enterobacterales; CRE), P. aeruginosa ATCC 27853 (WT), and S. aureus ATCC 29213 (WT). The corresponding MICs are shown in FIG. 9. For each organism, the anti-parallel D8 dimer exerts greater antimicrobial effects.

### Human Red Blood Cell Hemolysis

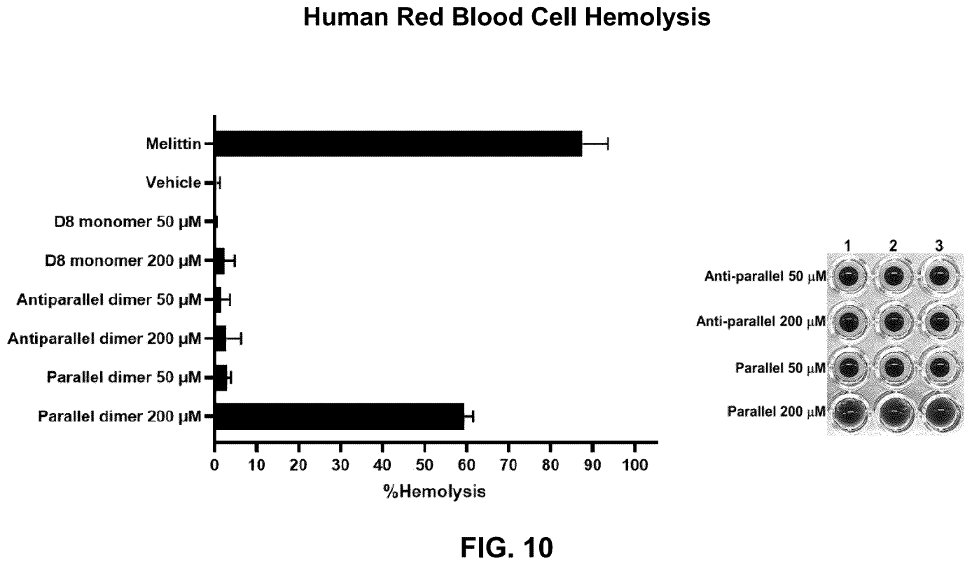

To assess potential hemolytic activity, the anti-parallel dimer (P201) and the parallel dimer (P261) were tested in a human red blood cell (RBC) hemolysis assay—a standard in vitro method that measures peptide-induced RBC rupture and hemoglobin release by spectrophotometry. Two concentrations of P201 and P261 (50 and 200 μM) were tested. The cytolytic peptide melittin and vehicle-alone served as positive and negative controls, respectively. Monomeric peptide D8 was used as a peptide control. As shown in FIG. 10, antimicrobial concentrations of the anti-parallel dimer did not demonstrate detectable hemolysis, similar to monomeric peptide D8. In contrast, the parallel dimer induced hemolytic activity in a dose-dependent manner.

### Human Cell Cytotoxicity

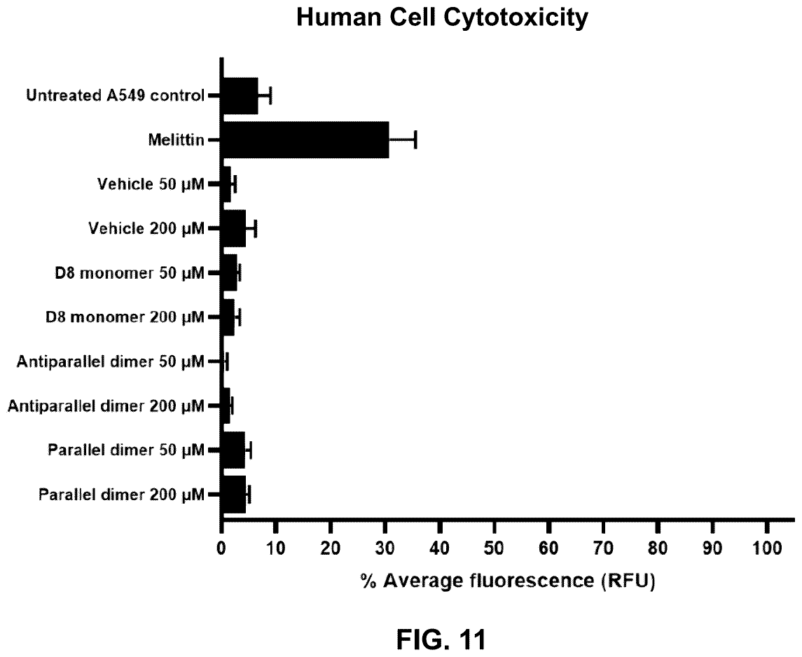

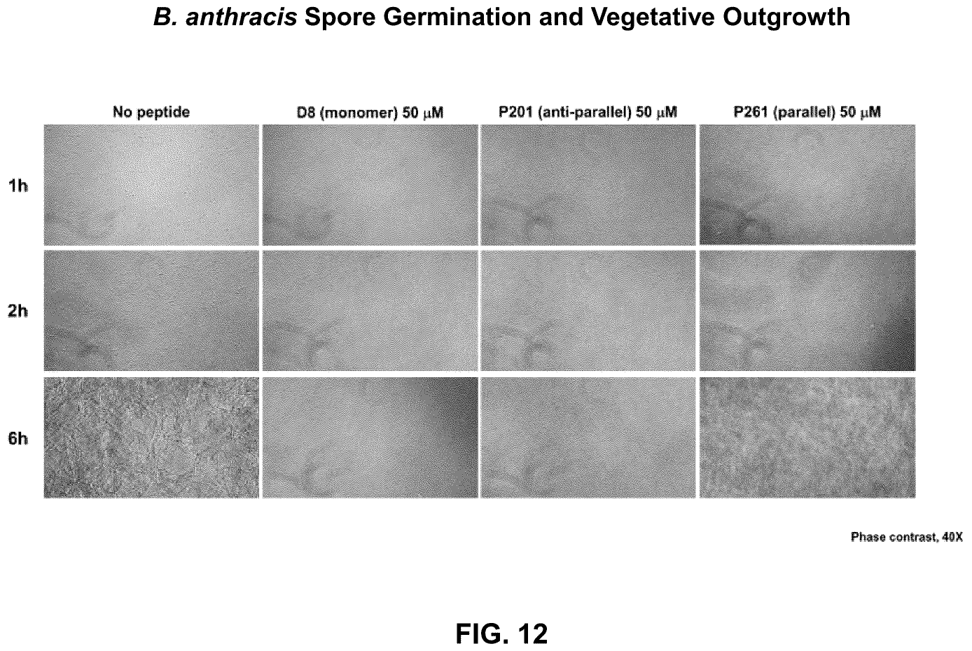

To evaluate potential cytotoxicity against human cells, the anti-parallel dimer (P201) and the parallel dimer (P261) were tested using the lung epithelial cell line A549 and a dye-based in vitro cytotoxicity assay where fluorescent signal is proportional to cell death. Two concentrations of P201 and P261 (50 and 200 μM) were tested. Melittin and vehicle-alone served as positive and negative controls, respectively. Monomeric peptide D8 was used as a peptide control. As displayed in FIG. 11, both concentrations of the anti-parallel dimer generated minimal fluorescence signal, comparable to monomeric D8, indicating little to no cytotoxicity. The parallel dimer P261 induced low-levels of fluorescence at both peptide concentrations tested, though not to the same extent as the cytolytic peptide melittin.

Bacillus anthracis Spore Germination and Vegetative Outgrowth

Bacillus anthracis is a spore-forming pathogen that causes anthrax. Spore germination marks the resumption of metabolic activity and is followed by the outgrowth of vegetative cells. Monomeric peptide D8 has demonstrated unique bactericidal efficacy against B. anthracis, appearing to interfere with both spore germination and vegetative outgrowth. To investigate whether the anti-parallel dimer (P201) and/or parallel dimer (P261) exhibit similar effects, B. anthracis Sterne strain spores were treated with 50 μM of individual peptides. The disruption of spore germination and bacterial viability were respectively assessed visually and by cfu determination+/−heat treatment, which allows the differentiation between heat-resistant spores and heat-sensitive bacilli.

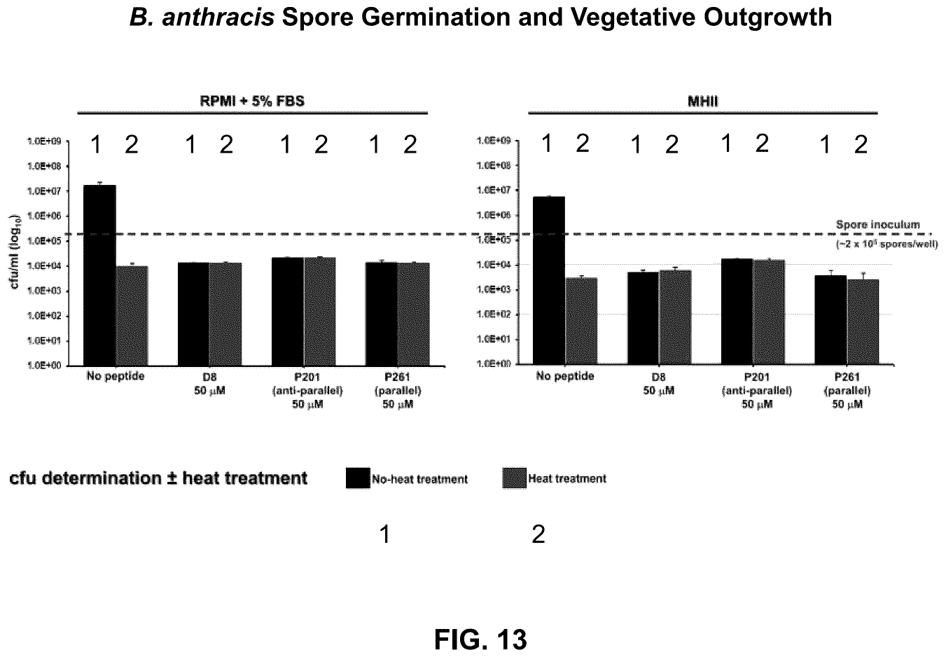

Representative phase-contrast images are shown in FIG. 12. The no-peptide control samples revealed spore germination and substantial vegetative outgrowth by 6 hours. The D8 monomer, anti-parallel dimer, and parallel dimer each exhibited antimicrobial effects, suppressing both spore germination and vegetative growth. Of note, a precipitate was observed to form in P261-treated samples by 6 h. Colony counts presented in FIG. 13, indicated that the D8 monomer, anti-parallel dimer, and parallel dimer P261 produced similar outcomes. Indeed, while the no-peptide control at 6 hours demonstrated spore germination and outgrowth, as indicated by a substantial loss of heat-resistant spores and an increase in heat-sensitive vegetative cells, the D8-, P201-, and P261-treated samples demonstrated reduced spore counts without any increase in vegetative cell numbers.

Labeled D8 Dimer MICs Against Klebsiella pneumoniae

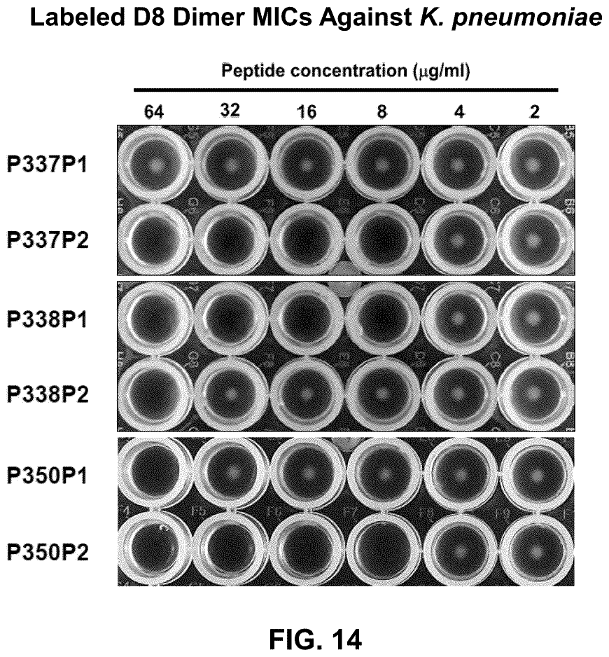

The incorporation of molecular labels into peptides enables experimental analyses to monitor molecular interactions with targets, investigate antimicrobial mechanisms, facilitate drug development, etc. To test the activities of D8 dimers with bioorthogonal “click-chemistry” labels, we performed unguided oxidation with D8 peptides containing an azide group (P337), a polyethylene glycol (PEG) azide group (P338), or an alkyne group (P350). As with unlabeled D8 in FIG. 2, the oxidation of each peptide resulted in the formation of two distinct peaks (P1 and P2) by HPLC analysis. The antimicrobial activities of products from both peaks, for each peptide, were determined against K. pneumoniae strain ATCC 43816 using the broth microdilution assay. As shown in FIG. 14, each peptide generated a more-active and less-active peak, presumably corresponding to the anti-parallel and parallel orientation, respectively. PEGylation appeared to change the elution order of the two possible dimers.

### REFERENCES

All references listed below and/or in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990) 215 J Mol Biol 403-410.
Ausubel et al. (2002) Short Protocols in Molecular Biology. Fifth ed. John Wylie & Sons, New York, New York, United States of America.
Ausubel et al. (2003) Current Protocols in Molecular Biology, John Wylie & Sons, Inc, New York, New York, United States of America.
Bird et al. (1988) Science 242:423-426.
Bowie et al. (1990) Science 247:1306-1310.
Chou & Fasman (1974) Biochemistry 13:222-245.
Chou & Fasman (1978) Ann Rev Biochem 47: 251-276.
Chou & Fasman (1979) Biophys J 26:367-384.
Crawford et al. (2023) 9 ACS Infect Dis 122-139.
Dayhoff et al. (in Atlas of Protein Sequence and Structure 1978, National Biomedical Research Foundation, Washington D.C., United States of America.
Goodman el al. (1981) Perspectives in Peptide Chemistry pages 283-294.
Harlow & Lane (1988) Antibodies: A Laboratory Manual, Cold Spring Harbor Laboratory Press, Cold Spring Harbor, New York, United States of America.
Harlow & Lane (1999) Using Antibodies: A Laboratory Manual, Cold Spring Harbor Laboratory Press, Cold Spring Harbor, New York, United States of America.
Henikoff & Henikoff (1992) 89 Proc Natl Acad Sci USA 10915-10919.
Houston et al. (1988) Proc Natl Acad Sci USA 85:5879-5883.
Johnson & Overington (1993) J Mol Biol 233:716-738.
Karlin & Altschul (1993) 90 Proc Natl Acad Sci USA 5873-5877.
Kyte & Doolittle (1982) J Mol Biol 157:105-132.
Needleman & Wunsch (1970) 48 J Mol Biol 443-453.
Nielsen et al. (1991) Science 254:1497-1500.
Pearson & Lipman (1988) Proc Natl Acad Sci USA 85:2444-2448.
Remington et al. (1975) Remington's Pharmaceutical Sciences, 15^{th} ed., Mack Pub. Co., Easton, Pennsylvania, United States of America.
Smith & Waterman (1981) 2 Adv Appl Math 482-489.
U.S. Patent Application Publication No. 2011/0218140.
U.S. Pat. Nos. 4,522,752; 4,554,101; 5,034,506.
Wiegand et al. (2008) 3 Nature Protocols 163-175.

### Informal Sequence Listing

TABLE 5
Exemplary Peptides with SEQ ID NOs.,
Sequences, and Peptide Names

| | | SEQ ID |
|---|---|---|
| Sequence | Peptide Name | NO: |
| RTVRCTCI | D8 | 1 |
| RAVRCTCI | D8-T2A | 2 |
| RRVRCTCI | D8-T2R | 3 |
| RNVRCTCI | D8-T2N | 4 |
| RDVRCTCI | D8-T2D | 5 |
| RCVRCTCI | D8-T2C | 6 |
| RQVRCTCI | D8-T2Q | 7 |
| REVRCTCI | D8-T2E | 8 |
| RHVRCTCI | D8-T2H | 9 |
| RIVRCTCI | D8-T2I | 10 |
| RLVRCTCI | D8-T2L | 11 |
| RKVRCTCI | D8-T2K | 12 |
| RMVRCTCI | D8-T2M | 13 |
| RFVRCTCI | D8-T2F | 14 |
| RPVRCTCI | D8-T2P | 15 |
| RGVRCTCI | D8-T2G | 16 |
| RSVRCTCI | D8-T2S | 17 |
| RWVRCTCI | D8-T2W | 18 |
| RYVRCTCI | D8-T2Y | 19 |
| RVVRCTCI | D8-T2V | 20 |
| R[alloThr]VRCTCI | D8-T2alloThr | 21 |
| R[MeThr]VRCTCI | D8-T2MeThr | 22 |
| R[Abu]VRCTCI | D8-T2Abu | 23 |
| R[NaphthylAla]VRCTCI | D8-T2NapAla | 24 |
| RTVRCACI | D8-T6A | 25 |
| RTVRCRCI | D8-T6R | 26 |
| RTVRCNCI | D8-T6N | 27 |
| RTVRCDCI | D8-T6D | 28 |
| RTVRCCCI | D8-T6C | 29 |
| RTVRCQCI | D8-T6Q | 30 |
| RTVRCECI | D8-T6E | 31 |
| RTVRCGCI | D8-T6G | 32 |
| RTVRCHCI | D8-T6H | 33 |
| RTVRCICI | D8-T6I | 34 |
| RTVRCLCI | D8-T6L | 35 |
| RTVRCKCI | D8-T6K | 36 |
| RTVRCMCI | D8-T6M | 37 |
| RTVRCFCI | D8-T6F | 38 |
| RTVRCPCI | D8-T6P | 39 |
| RTVRCSCI | D8-T6S | 40 |
| RTVRCWCI | D8-T6W | 41 |
| RTVRCYCI | D8-T6Y | 42 |
| RTVRCVCI | D8-T6V | 43 |
| RTVRC[alloThr]CI | D8-T6alloThr | 44 |
| RTVRC[MeThr]CI | D8-T6MeThr | 45 |
| RTVRC[Abu]CI | D8-T6Abu | 46 |
| RTVRC[NaphthylAla]CI | D8-T6NapAla | 47 |
| RTVRC[Phe(3-Me)]CI | D8-T6Phe(3-Me) | 48 |
| RTVRC [Bip]CI | D8-T6Bip | 49 |
| Pra-RTVRCTCI | Pra-D8 | 50 |
| Ala(N3)-RTVRCTCI | Ala(N3)-D8 | 51 |
| PEG2-RTVRCTCI | PEG2-D8 | 52 |
| PEG2(N3)-RTVRCTCI | PEG2(N3)-D8 | 53 |
| PEG4-RTVRCTCI | PEG4-D8 | 54 |
| RTVRCTCI-Pra | D8-Pra | 55 |
| RTVRCTCI-Ala(N3) | D8-Ala(N3) | 56 |
| RTVRCTCI-PEG2(N3) | D8-PEG2(N3) | 57 |
| RTVR[hC]T[hC]I | D8-C5hC, C7hC | 58 |
| RYVRCRCI | P194 | 59 |
| AYVRCRCI | P194-R1A | 60 |
| NYVRCRCI | P194-RIN | 61 |
| DYVRCRCI | P194-R1D | 62 |
| CYVRCRCI | P194-R1C | 63 |
| QYVRCRCI | P194-R1Q | 64 |
| EYVRCRCI | P194-RIE | 65 |
| GYVRCRCI | P194-R1G | 66 |
| HYVRCRCI | P194-R1H | 67 |
| IYVRCRCI | P194-R1I | 68 |
| LYVRCRCI | P194-R1L | 69 |
| KYVRCRCI | P194-R1K | 70 |
| MYVRCRCI | P194-R1M | 71 |
| FYVRCRCI | P194-RIF | 72 |
| PYVRCRCI | P194-RIP | 73 |
| SYVRCRCI | P194-RIS | 74 |
| TYVRCRCI | P194-R1T | 75 |
| WYVRCRCI | P194-R1W | 76 |
| YYVRCRCI | P194-R1Y | 77 |
| VYVRCRCI | P194-R1V | 78 |
| [Cit]YVRCRCI | P194-R1Cit | 79 |
| [hR]YVRCRCI | P194-R1hR | 80 |
| [R(NO2)]YVRCRCI | P194-RIR(NO₂) | 81 |
| [Agb]YVRCRCI | P194-R1Agb | 82 |
| RYVRCRCI | P194 | 83 |
| RAVRCRCI | P194-Y2A | 84 |
| RRVRCRCI | P194-Y2R | 85 |
| RNVRCRCI | P194-Y2N | 86 |
| RDVRCRCI | P194-Y2D | 87 |
| RCVRCRCI | P194-Y2C | 88 |
| RQVRCRCI | P194-Y2Q | 89 |
| REVRCRCI | P194-Y2E | 90 |
| RGVRCRCI | P194-Y2G | 91 |
| RHVRCRCI | P194-Y2H | 92 |
| RIVRCRCI | P194-Y2I | 93 |
| RLVRCRCI | P194-Y2L | 94 |
| RKVRCRCI | P194-Y2K | 95 |
| RMVRCRCI | P194-Y2M | 96 |
| RFVRCRCI | P194-Y2F | 97 |
| RPVRCRCI | P194-Y2P | 98 |
| RSVRCRCI | P194-Y2S | 99 |
| RTVRCRCI | P194-Y2T | 100 |
| RWVRCRCI | P194-Y2W | 101 |
| RVVRCRCI | P194-Y2V | 102 |
| R[MeTyr]VRCRCI | P194-Y2MeTyr | 103 |
| R[DimethylTyr]VRCRCI | P194-Y2DiMethylTyr | 104 |
| R[hP]VRCRCI | P194-Y2hP | 105 |
| PEG2-RYVRCRCI | PEG2-P194 | 106 |
| PEG4-RYVRCRCI | PEG4-P194 | 107 |
| RWVRCWCI | RWVRCWCI | 108 |
| LSRTVRCTCISI | LSRTVRCTCISI | 109 |
| VPLSRTVRCTCISI | VPLSRTVRCTCISI | 110 |
| ICTCRVTR | Reverse Sequence | 111 |
| | of SEQ ID NO: 1 | |

Abu: aminobutyric acid; Agb: norarginine; Ala(N3): azido alanine; alloThr: allothreonine; Bip: 3-(4-biphenyl) alanine; Cit: citrulline; DimethylTyr: 2,6-dimethyl tyrosine; hC: homocysteine; hP: homoproline; hR: homoarginine; MeThr: O-methyl threonine; MeTyr: O-methyl tyrosine; NaphthylAla: 2- naphthyl alanine; PEG2: two PEG (polyethylene glycol) groups; PEG2(N3): azido PEG2; PEG4: four PEG groups; Phe(3-Me): 3-methyl phenylalanine; Pra: propargylglycine; R(NO₂): nitroarginine. In certain aspects each amino of each sequence above can be a D-amino acid. In certain embodiments, each sequence above can comprise one or more D-amino acids. In some embodiments, each sequence above can comprise one or more D-amino acids, except in positions where the amino acid is a modified amino acid. In some embodiments, each amino acid at each position is a D-amino acid except where there is a modified amino acid.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A peptide dimer, comprising:
a first peptide and a second peptide, wherein:
the first and second peptides both comprise one or more of RTVRCTCI (SEQ ID NO: 1), RFVRCTCI (SEQ ID NO: 14), RWVRCTCI (SEQ ID NO: 18), RYVRCTCI (SEQ ID NO: 19), RTVRCRCI (SEQ ID NO: 26), RTVRCMCI (SEQ ID NO: 37), RYVRCRCI (SEQ ID NO: 59), RRVRCRCI (SEQ ID NO: 85), RWVRCWCI (SEQ ID NO: 108), or modified peptides thereof, wherein the modified peptides have at least one cysteine and comprise one or more amino acid substitutions relative to SEQ ID NOs: 1, 14, 18, 19, 26, 37, 59, 85, or 108.

2. The peptide dimer of claim 1, wherein the first and second peptides are present in a parallel or an anti-parallel orientation.

3. The peptide dimer of claim 1, wherein one or more of the amino acids of the first peptide, the second peptide, or both, are D-amino acids.

4. The peptide dimer of claim 1, wherein all of the amino acids of the first peptide, the second peptide, or both, are D-amino acids.

5. The peptide dimer of claim 1, wherein the first peptide, the second peptide, or both, comprises the amino acid sequence RTVRCTCI (SEQ ID NO: 1), RTVRCRCI (SEQ ID NO: 26), RTVRCMCI (SEQ ID NO: 37), or RYVRCRCI (SEQ ID NO: 59), or a modified amino acid sequence thereof, wherein the modified amino acid sequence comprises at least one or two cysteines and one or more amino acid substitutions relative to SEQ ID NOs: 1, 26, 37, or 59; and wherein one or more of the amino acids of the first peptide, the second peptide, or both, are D-amino acids.

6. The peptide dimer of claim 1, wherein the first peptide, the second peptide, or both, is polymer-functionalized, encapsulated in a particle, embedded in a solid support, on a solid support, or any combination of any thereof.

7. The peptide dimer of claim 1, wherein the peptide dimer is formulated for release from a solid support, impregnated in a dressing and formulated for release from the dressing, formulated for use in a nebulizer, formulated for use in a topical administration, or formulated for use for systemic administration, individually or in any combination of any thereof.

8. The peptide dimer of claim 1, wherein the first peptide, the second peptide, or both, is a modified peptide that comprises a modification: at the N-terminus, at the C-terminus, or both the N-terminus and the C-terminus, or at an internal amino acid position including but not limited to amino acid position 2, individually or in any combination of any thereof.

9. The peptide dimer of claim 7, wherein the one or more of the modifications are one or more of an addition or an aminohexanoic acid (AHX), an azido alanine (Ala(N₃)), a propargylglycine (PRA), a photoaffinity label, a benzoylbenzoic acid (4-BBA), an azido phenylalanine (Phe(4-N₃)), a diazirine-containing amino acid, leucine, methionine, lysine, proline, phenylalanine, or is a replacement of a phenylalanine with an azido phenylalanine (Phe(4-N₃)), individually or in any combination of any thereof.

10. A pharmaceutical composition comprising:
a peptide dimer of claim 1; and
a pharmaceutically acceptable carrier, diluent, or excipient.

11. A medical device comprising a support layer with an antibacterial agent embedded therein or associated therewith, wherein the antibacterial agent comprises the peptide dimer of claim 1, optionally wherein the medical device is a wound dressing.

12. The medical device of claim 11, wherein the medical device is a wound dressing.

13. The medical device of claim 11, wherein the peptide dimer is encapsulated in a particle that is embedded in or associated with the support layer.

14. A method for inhibiting the growth of, killing a bacterium, or both, the method comprising: contacting the bacterium with an effective amount of an antibacterial agent comprising one or more peptide dimers of claim 1.

15. The method of claim 14, wherein the bacterium is one or more of Enterococcus faecium, Staphylococcus aureus, Klebsiella pneumoniae, Acinetobacter baumannii, Pseudomonas aeruginosa, members of the family Enterobacteriaceae, one or more sexually-transmitted bacteria, an enteric pathogen, or a biothreat agent in vegetative forms, spore forms, or both, individually or in any combination of any thereof.

16. The method of claim 15, wherein the bacterium is a multi-drug resistant (MDR) strain of Enterococcus faecium, Staphylococcus aureus, Klebsiella pneumoniae, Acinetobacter baumannii, Pseudomonas aeruginosa, Salmonella enterica, optionally Salmonella enterica serovar Typhi, or Shigella flexneri, individually or in any combination thereof.

17. The method of claim 14, wherein the bacterium is present in or on a subject having or suspected of having a community infection, a nosocomial infection, a wound infected by bacteria, a pulmonary infection, a sexually-transmitted bacterial infection, exposure to a biothreat agent, or a systemic infection, individually or in any combination thereof.

18. The method of claim 17, further comprising administering to the subject a conventional antibiotic.

19. A method for inhibiting the growth of a biofilm, comprising:
contacting the biofilm with an effective amount of an antibacterial agent comprising one or more peptide dimers of claim 1.


SEQUENCE LISTING (111 sequences)

SEQ ID NO: 1 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 2 - protein, 8 aa, synthetic construct
  SITE 1: note = D-Arginine
  SITE 2: note = D-Alanine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RAVRCTCI

SEQ ID NO: 3 - protein, 8 aa, synthetic construct
  SITE 1..2: note = D-Arginine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RRVRCTCI

SEQ ID NO: 4 - protein, 8 aa, synthetic construct
  SITE 1: note = D-Arginine
  SITE 2: note = D-Asparagine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RNVRCTCI

SEQ ID NO: 5 - protein, 8 aa, synthetic construct
  SITE 1: note = D-Arginine
  SITE 2: note = D-Aspartic acid
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RDVRCTCI

SEQ ID NO: 6 - protein, 8 aa, synthetic construct
  SITE 1: note = D-Arginine
  SITE 2: note = D-Cysteine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RCVRCTCI

SEQ ID NO: 7 - protein, 8 aa, synthetic construct
  SITE 1: note = D-Arginine
  SITE 2: note = D-Glutamine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RQVRCTCI

SEQ ID NO: 8 - protein, 8 aa, synthetic construct
  SITE 1: note = D-Arginine
  SITE 2: note = D-Glutamic acid
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
REVRCTCI

SEQ ID NO: 9 - protein, 8 aa, synthetic construct
  SITE 1: note = D-Arginine
  SITE 2: note = D-Histidine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RHVRCTCI

SEQ ID NO: 10 - protein, 8 aa, synthetic construct
  SITE 1: note = D-Arginine
  SITE 2: note = D-Isoleucine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RIVRCTCI

SEQ ID NO: 11 - protein, 8 aa, synthetic construct
  SITE 1: note = D-Arginine
  SITE 2: note = D-Leucine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RLVRCTCI

SEQ ID NO: 12 - protein, 8 aa, synthetic construct
  SITE 1: note = D-Arginine
  SITE 2: note = D-Lysine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RKVRCTCI

SEQ ID NO: 13 - protein, 8 aa, synthetic construct
  SITE 1: note = D-Arginine
  SITE 2: note = D-Methionine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RMVRCTCI

SEQ ID NO: 14 - protein, 8 aa, synthetic construct
  SITE 1: note = D-Arginine
  SITE 2: note = D-Phenylalanine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RFVRCTCI

SEQ ID NO: 15 - protein, 8 aa, synthetic construct
  SITE 1: note = D-Arginine
  SITE 2: note = D-Proline
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RPVRCTCI

SEQ ID NO: 16 - protein, 8 aa, synthetic construct
  SITE 1: note = D-Arginine
  SITE 2: note = D-Glycine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RGVRCTCI

SEQ ID NO: 17 - protein, 8 aa, synthetic construct
  SITE 1: note = D-Arginine
  SITE 2: note = D-Serine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RSVRCTCI

SEQ ID NO: 18 - protein, 8 aa, synthetic construct
  SITE 1: note = D-Arginine
  SITE 2: note = D-Tryptophan
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RWVRCTCI

SEQ ID NO: 19 - protein, 8 aa, synthetic construct
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RYVRCTCI

SEQ ID NO: 20 - protein, 8 aa, synthetic construct
  SITE 1: note = D-Arginine
  SITE 2..3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RVVRCTCI

SEQ ID NO: 21 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
  MOD_RES 2: note = Allothreonine
RTVRCTCI

SEQ ID NO: 22 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
  MOD_RES 2: note = O-methyl threonine
RTVRCTCI

SEQ ID NO: 23 - protein, 8 aa, synthetic construct
  MOD_RES 2: note = Aminobutyric acid
  SITE 1: note = D-Arginine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RXVRCTCI

SEQ ID NO: 24 - protein, 8 aa, synthetic construct
  MOD_RES 2: note = 2-naphthyl alanine
  SITE 1: note = D-Arginine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RXVRCTCI

SEQ ID NO: 25 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RTVRCACI

SEQ ID NO: 26 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RTVRCRCI

SEQ ID NO: 27 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Asparagine
  SITE 7: note = D-Cysteine
  SITE 8: note = D-Isoleucine
RTVRCNCI

SEQ ID NO: 28 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Aspartic acid
  SITE 7: note = D-Cysteine
  SITE 8: note = D-Isoleucine
RTVRCDCI

SEQ ID NO: 29 - protein, 8 aa, synthetic construct
  SITE 1: note = D-Arginine
  SITE 2: note = D-Threonine
  SITE 3: note = D-Valine
  SITE 4: note = D-Arginine
  SITE 5..7: note = D-Cysteine
  SITE 8: note = D-Isoleucine
RTVRCCCI

SEQ ID NO: 30 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Glutamine
  SITE 7: note = D-Cysteine
  SITE 8: note = D-Isoleucine
RTVRCQCI

SEQ ID NO: 31 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Glutamic acid
  SITE 7: note = D-Cysteine
  SITE 8: note = D-Isoleucine
RTVRCECI

SEQ ID NO: 32 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Glycine
  SITE 7: note = D-Cysteine
  SITE 8: note = D-Isoleucine
RTVRCGCI

SEQ ID NO: 33 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Histidine
  SITE 7: note = D-Cysteine
  SITE 8: note = D-Isoleucine
RTVRCHCI

SEQ ID NO: 34 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Isoleucine
  SITE 7: note = D-Cysteine
  SITE 8: note = D-Isoleucine
RTVRCICI

SEQ ID NO: 35 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Leucine
  SITE 7: note = D-Cysteine
  SITE 8: note = D-Isoleucine
RTVRCLCI

SEQ ID NO: 36 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Lysine
  SITE 7: note = D-Cysteine
  SITE 8: note = D-Isoleucine
RTVRCKCI

SEQ ID NO: 37 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Methionine
  SITE 7: note = D-Cysteine
  SITE 8: note = D-Isoleucine
RTVRCMCI

SEQ ID NO: 38 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Phenylalanine
  SITE 7: note = D-Cysteine
  SITE 8: note = D-Isoleucine
RTVRCFCI

SEQ ID NO: 39 - protein, 8 aa, synthetic construct
RTVRCPCI

SEQ ID NO: 40 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Serine
  SITE 7: note = D-Cysteine
  SITE 8: note = D-Isoleucine
RTVRCSCI

SEQ ID NO: 41 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Tryptophan
  SITE 7: note = D-Cysteine
  SITE 8: note = D-Isoleucine
RTVRCWCI

SEQ ID NO: 42 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Tyrosine
  SITE 7: note = D-Cysteine
  SITE 8: note = D-Isoleucine
RTVRCYCI

SEQ ID NO: 43 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Valine
  SITE 7: note = D-Cysteine
  SITE 8: note = D-Isoleucine
RTVRCVCI

SEQ ID NO: 44 - protein, 8 aa, synthetic construct
  MOD_RES 6: note = Allothreonine
RTVRCTCI

SEQ ID NO: 45 - protein, 8 aa, synthetic construct
  MOD_RES 6: note = O-methyl threonine
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 46 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  MOD_RES 6: note = Aminobutyric acid
  SITE 7: note = D-Cysteine
  SITE 8: note = D-Isoleucine
RTVRCXCI

SEQ ID NO: 47 - protein, 8 aa, synthetic construct
  MOD_RES 6: note = 2-naphthyl alanine
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Alanine
  SITE 7: note = D-Cysteine
  SITE 8: note = D-Isoleucine
RTVRCACI

SEQ ID NO: 48 - protein, 8 aa, synthetic construct
  MOD_RES 6: note = 3-methyl phenylalanine
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Phenylalanine
  SITE 7: note = D-Cysteine
  SITE 8: note = D-Isoleucine
RTVRCFCI

SEQ ID NO: 49 - protein, 8 aa, synthetic construct
  MOD_RES 6: note = 3-(4-biphenyl) alanine
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Alanine
  SITE 7: note = D-Cysteine
  SITE 8: note = D-Isoleucine
RTVRCACI

SEQ ID NO: 50 - protein, 8 aa, synthetic construct
  MOD_RES 1: note = R at position 1 is propargylglycine modified
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 51 - protein, 8 aa, synthetic construct
  MOD_RES 1: note = R at position 1 is azido alanine-modified
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 52 - protein, 8 aa, synthetic construct
  MOD_RES 1: note = R at position 1 is modified with two PEG (polyethylene glycol) groups
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 53 - protein, 8 aa, synthetic construct
  MOD_RES 1: note = R at position 1 is azido PEG2 modified
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 54 - protein, 8 aa, synthetic construct
  MOD_RES 1: note = R at position 1 is modified with four PEG groups
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 55 - protein, 8 aa, synthetic construct
  MOD_RES 8: note = I at position 8 is propargylglycine-modified
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 56 - protein, 8 aa, synthetic construct
  MOD_RES 8: note = I at position 8 is azido alanine modified
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 57 - protein, 8 aa, synthetic construct
  MOD_RES 8: note = I at position 8 is azido PEG2 modified
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Threonine
  SITE 7: note = D-Cysteine
  SITE 8: note = D-Isoleucine
RTVRCTCI

SEQ ID NO: 58 - protein, 10 aa, synthetic construct
  MOD_RES 5: note = Homocysteine
  MOD_RES 7: note = Homocysteine
  SITE 1: note = D-Arginine
  SITE 2: note = D-Threonine
  SITE 3: note = D-Valine
  SITE 4: note = D-Arginine
  SITE 5: note = D-Histidine
  SITE 6: note = D-Cysteine
  SITE 7: note = D-Threonine
  SITE 8: note = D-Histidine
  SITE 9: note = D-Cysteine
  SITE 10: note = D-Isoleucine
RTVRHCTHCI

SEQ ID NO: 59 - protein, 8 aa, synthetic construct
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 60 - protein, 8 aa, synthetic construct
  SITE 1: note = D-Alanine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
AYVRCRCI

SEQ ID NO: 61 - protein, 8 aa, synthetic construct
  SITE 1: note = D-Asparagine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
NYVRCRCI

SEQ ID NO: 62 - protein, 8 aa, synthetic construct
  SITE 1: note = D-Aspartic acid
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
DYVRCRCI

SEQ ID NO: 63 - protein, 8 aa, synthetic construct
  SITE 1: note = D-Cysteine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
CYVRCRCI

SEQ ID NO: 64 - protein, 8 aa, synthetic construct
  SITE 1: note = D-Glutamine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
QYVRCRCI

SEQ ID NO: 65 - protein, 8 aa, synthetic construct
  SITE 1: note = D-Glutamic acid
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
EYVRCRCI

SEQ ID NO: 66 - protein, 8 aa, synthetic construct
  SITE 1: note = D-Glycine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
GYVRCRCI

SEQ ID NO: 67 - protein, 8 aa, synthetic construct
  SITE 1: note = D-Histidine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
HYVRCRCI

SEQ ID NO: 68 - protein, 8 aa, synthetic construct
  SITE 1: note = D-Isoleucine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
IYVRCRCI

SEQ ID NO: 69 - protein, 8 aa, synthetic construct
  SITE 1: note = D-Leucine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
LYVRCRCI

SEQ ID NO: 70 - protein, 8 aa, synthetic construct
  SITE 1: note = D-Lysine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
KYVRCRCI

SEQ ID NO: 71 - protein, 8 aa, synthetic construct
  SITE 1: note = D-Methionine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
MYVRCRCI

SEQ ID NO: 72 - protein, 8 aa, synthetic construct
  SITE 1: note = D-Phenylalanine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
FYVRCRCI

SEQ ID NO: 73 - protein, 8 aa, synthetic construct
  SITE 1: note = D-Proline
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
PYVRCRCI

SEQ ID NO: 74 - protein, 8 aa, synthetic construct
  SITE 1: note = D-Serine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
SYVRCRCI

SEQ ID NO: 75 - protein, 8 aa, synthetic construct
  SITE 2: note = D-Tyrosine
  SITE 1: note = D-Threonine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
TYVRCRCI

SEQ ID NO: 76 - protein, 8 aa, synthetic construct
  SITE 1: note = D-Tryptophan
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
WYVRCRCI

SEQ ID NO: 77 - protein, 8 aa, synthetic construct
  SITE 1..2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
YYVRCRCI

SEQ ID NO: 78 - protein, 8 aa, synthetic construct
  SITE 1: note = D-Valine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
VYVRCRCI

SEQ ID NO: 79 - protein, 8 aa, synthetic construct
  MOD_RES 1: note = Citrulline
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
XYVRCRCI

SEQ ID NO: 80 - protein, 8 aa, synthetic construct
  MOD_RES 1: note = Homoarginine
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 81 - protein, 8 aa, synthetic construct
  MOD_RES 1: note = Nitroarginine
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 82 - protein, 8 aa, synthetic construct
  MOD_RES 1: note = Norarginine
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 83 - protein, 8 aa, synthetic construct
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 84 - protein, 8 aa, synthetic construct
  SITE 1: note = D-Arginine
  SITE 2: note = D-Alanine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RAVRCRCI

SEQ ID NO: 85 - protein, 8 aa, synthetic construct
  SITE 1..2: note = D-Arginine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RRVRCRCI

SEQ ID NO: 86 - protein, 8 aa, synthetic construct
  SITE 1: note = D-Arginine
  SITE 2: note = D-Asparagine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RNVRCRCI

SEQ ID NO: 87 - protein, 8 aa, synthetic construct
  SITE 1: note = D-Arginine
  SITE 2: note = D-Aspartic acid
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RDVRCRCI

SEQ ID NO: 88 - protein, 8 aa, synthetic construct
  SITE 1: note = D-Arginine
  SITE 2: note = D-Cysteine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RCVRCRCI

SEQ ID NO: 89 - protein, 8 aa, synthetic construct
  SITE 1: note = D-Arginine
  SITE 2: note = D-Glutamine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RQVRCRCI

SEQ ID NO: 90 - protein, 8 aa, synthetic construct
  SITE 1: note = D-Arginine
  SITE 2: note = D-Glutamic acid
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
REVRCRCI

SEQ ID NO: 91 - protein, 8 aa, synthetic construct
  SITE 1: note = D-Arginine
  SITE 2: note = D-Glycine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RGVRCRCI

SEQ ID NO: 92 - protein, 8 aa, synthetic construct
  SITE 1: note = D-Arginine
  SITE 2: note = D-Histidine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RHVRCRCI

SEQ ID NO: 93 - protein, 8 aa, synthetic construct
  SITE 1: note = D-Arginine
  SITE 2: note = D-Isoleucine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RIVRCRCI

SEQ ID NO: 94 - protein, 8 aa, synthetic construct
  SITE 1: note = D-Arginine
  SITE 2: note = D-Leucine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RLVRCRCI

SEQ ID NO: 95 - protein, 8 aa, synthetic construct
  SITE 1: note = D-Arginine
  SITE 2: note = D-Lysine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RKVRCRCI

SEQ ID NO: 96 - protein, 8 aa, synthetic construct
  SITE 1: note = D-Arginine
  SITE 2: note = D-Methionine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RMVRCRCI

SEQ ID NO: 97 - protein, 8 aa, synthetic construct
  SITE 1: note = D-Arginine
  SITE 2: note = D-Phenylalanine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RFVRCRCI

SEQ ID NO: 98 - protein, 8 aa, synthetic construct
  SITE 1: note = D-Arginine
  SITE 2: note = D-Proline
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RPVRCRCI

SEQ ID NO: 99 - protein, 8 aa, synthetic construct
  SITE 1: note = D-Arginine
  SITE 2: note = D-Serine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RSVRCRCI

SEQ ID NO: 100 - protein, 8 aa, synthetic construct
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RTVRCRCI

SEQ ID NO: 101 - protein, 8 aa, synthetic construct
  SITE 1: note = D-Arginine
  SITE 2: note = D-Tryptophan
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RWVRCRCI

SEQ ID NO: 102 - protein, 8 aa, synthetic construct
  SITE 1: note = D-Arginine
  SITE 2..3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RVVRCRCI

SEQ ID NO: 103 - protein, 8 aa, synthetic construct
  MOD_RES 2: note = O-methyl tyrosine
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RTVRCRCI

SEQ ID NO: 104 - protein, 8 aa, synthetic construct
  MOD_RES 2: note = 2,6-dimethyl tyrosine
  SITE 1: note = D-Arginine
  SITE 2: note = D-Threonine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RTVRCRCI

SEQ ID NO: 105 - protein, 8 aa, synthetic construct
  MOD_RES 2: note = Homoproline
  SITE 1: note = D-Arginine
  SITE 2: note = D-Proline
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RPVRCRCI

SEQ ID NO: 106 - protein, 8 aa, synthetic construct
  MOD_RES 1: note = R at position 1 is modified with 2 PEG groups
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 107 - protein, 8 aa, synthetic construct
  MOD_RES 1: note = R at position 1 is modified with 4 PEG groups
  SITE 1: note = D-Arginine
  SITE 2: note = D-Tyrosine
  SITE 3: note = D-Valine
  SITE 4: note = D-Arginine
  SITE 5: note = D-Cysteine
  SITE 6: note = D-Arginine
  SITE 7: note = D-Cysteine
  SITE 8: note = D-Isoleucine
RYVRCRCI

SEQ ID NO: 108 - protein, 8 aa, synthetic construct
  SITE 1: note = D-Arginine
  SITE 2: note = D-Tryptophan
  SITE 3: note = D-Valine
  SITE 4: note = D-Arginine
  SITE 5: note = D-Cysteine
  SITE 6: note = D-Tryptophan
  SITE 7: note = D-Cysteine
  SITE 8: note = D-Isoleucine
RWVRCWCI

SEQ ID NO: 109 - protein, 12 aa, synthetic construct
  SITE 1: note = D-Leucine
  SITE 2: note = D-Serine
  SITE 3: note = D-Arginine
  SITE 4: note = D-Threonine
  SITE 5: note = D-Valine
  SITE 6: note = D-Arginine
  SITE 7: note = D-Cysteine
  SITE 8: note = D-Threonine
  SITE 9: note = D-Cysteine
  SITE 10: note = D-Isoleucine
  SITE 11: note = D-Serine
  SITE 12: note = D-Isoleucine
LSRTVRCTCISI

SEQ ID NO: 110 - protein, 14 aa, synthetic construct
  SITE 1: note = D-Valine
  SITE 2: note = D-Proline
  SITE 3: note = D-Leucine
  SITE 4: note = D-Serine
  SITE 5: note = D-Arginine
  SITE 6: note = D-Threonine
  SITE 7: note = D-Valine
  SITE 8: note = D-Arginine
  SITE 9: note = D-Cysteine
  SITE 10: note = D-Threonine
  SITE 11: note = D-Cysteine
  SITE 12: note = D-Isoleucine
  SITE 13: note = D-Serine
  SITE 14: note = D-Isoleucine
VPLSRTVRCTCISI

SEQ ID NO: 111 - protein, 8 aa, synthetic construct
  SITE 1: note = D-Isoleucine
  SITE 2: note = D-Cysteine
  SITE 3: note = D-Threonine
  SITE 4: note = D-Cysteine
  SITE 5: note = D-Arginine
  SITE 6: note = D-Valine
  SITE 7: note = D-Threonine
  SITE 8: note = D-Arginine
ICTCRVTR